SEQUENCE LISTING (14 sequences)

SEQ ID NO: 1 - protein, 214 aa, synthetic construct
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEK

SEQ ID NO: 2 - protein, 214 aa, synthetic construct
MSKGEELFTGVVPILVELDGDVNGHKFSVRGEGEGDATIGKLTLKFICTTGKLPVPWPTLVTTLTYGVQCFSRYPDHMKRHDFFKSAMPEGYVQERTISFKDDGKYKTRAVVKFEGDTLVNRIELKGTDFKEDGNILGHKLEYNFNSHNVYITADKQKNGIKANFTVRHNVEDGSVQLADHYQQNTPIGDGPVLLPDNHYLSTQTVLSKDPNEK

SEQ ID NO: 3 - protein, 19 aa, synthetic construct
KRDHMVLLEFVTAAGITGT

SEQ ID NO: 4 - protein, 19 aa, synthetic construct
KRDHMVLHEFVTAAGITGT

SEQ ID NO: 5 - protein, 17 aa, synthetic construct
KRDHMVLHESVNAAGIT

SEQ ID NO: 6 - protein, 16 aa, synthetic construct
RDHMVLHEYVNAAGIT

SEQ ID NO: 7 - protein, 17 aa, synthetic construct
GDAVQIQEHAVAKYFTV

SEQ ID NO: 8 - protein, 17 aa, synthetic construct
GDTVQLQEHAVAKYFTV

SEQ ID NO: 9 - protein, 17 aa, synthetic construct
GETIQLQEHAVAKYFTE

SEQ ID NO: 10 - protein, 208 aa, synthetic construct
MEEDNMAIIKEFMRFKVHMEGSVNGHEFEIEGEGEGHPYEGTQTAKLKVTKGGPLPFAWDILSPQFMYGSKAYVKHPADIPDYLKLSFPEGFTWERVMNFEDGGVVTVTQDSSLQDGEFIYKVKLLGTNFPSDGPVMQKKTMGWEASTERMYPEDGALKGEINQRLKLKDGGHYDAEVKTTYKAKKPVQLPGAYNVDIKLDITSHNED

SEQ ID NO: 11 - protein, 18 aa, synthetic construct
YTIVEQYERAEGRHSTGG

SEQ ID NO: 12 - protein, 114 aa, synthetic construct
MEHVAFGSEDIENTLAKMDDGQLDGLAFGAIQLDGDGNILQYNAAEGDITGRDPKQVIGKNFFKDVAPGTDSPEFYGKFKEGVASGNLNTMFEWMIPTSRGPTKVKVHMKKALS

SEQ ID NO: 13 - protein, 11 aa, synthetic construct
GDSYWVFVKRV

SEQ ID NO: 14 - protein, 10 aa, synthetic construct
GDSYWVFVKR

(19) United States
(12) Patent Application Publication
(10) Pub. No.: US 2024/0359181 A1
(43) Pub. Date: Oct. 31, 2024

(54) METHODS AND COMPOSITIONS FOR IMPROVED BIOMOLECULE ASSAYS ON DIGITAL MICROFLUIDIC DEVICES

(71) Applicant: NUCLERA LTD, Cambridge (GB)

(72) Inventors: Michael Chun Hao Chen, Cambridge (GB); Sihong Chen, Cambridge (GB); Chiara Gandini, Cambridge (GB); Atanas Yordanov Georgiev, Cambridge (GB); Michal Jan Horka, Cambridge (GB); Sumit Kalsi, Cambridge (GB); Johnathon Lilley, Cambridge (GB); Richard J. Paolini, JR., Billerica, MA (US); Stephanie Reikine, Cambridge (GB); Luke M. Slominski, Billerica, MA (US)

(21) Appl. No.: 18/684,235

(22) PCT Filed: Aug. 18, 2022
(86) PCT No.: PCT/GB2022/052141

§ 371 Date: Feb. 16, 2024

(30) Foreign Application Priority Data
Aug. 18, 2021 (WO) PCT/GB2021/052140
Mar. 14, 2022 (GB) 2203522.4

(51) Int. Cl. B01L 3/00

(52) U.S. Cl. CPC B01L 3/502761; B01L 2200/16; B01L 2300/0645

(57) ABSTRACT

Provided herein are methods, and compositions for the detection and analysis of biomolecule interactions a microfluidic device. The detection and analysis occurs in aqueous droplets having a first surfactant within an oil layer having a second surfactant.

### FIELD OF THE INVENTION

Provided herein are methods and compositions for improved biomolecule assays on digital microfluidic devices. Provided herein are methods, and compositions for on-device protein synthesis and detection thereof. The methods are applicable to monitoring on a microfluidic device.

### BACKGROUND TO THE INVENTION

Electrowetting is the modification of the wetting properties of a surface (which is typically hydrophobic) with an applied electric field. Microfluidic devices for manipulating droplets or magnetic beads based on electrowetting have been extensively described. In the case of droplets in channels this can be achieved by causing the droplets, for example in the presence of an immiscible carrier fluid, to travel through a microfluidic channel defined by the walls of a cartridge or microfluidic tubing. Embedded in the walls of the cartridge or tubing are electrodes covered with a dielectric layer each of which are connected to an A/C biasing circuit capable of being switched on and off rapidly at intervals to modify the electrowetting field characteristics of the layer. This gives rise to the ability to steer the droplet along a given path.

As an alternative to microfluidic channel systems, droplets can also be generated and manipulated on planar surfaces using digital microfluidics (DMF). In contrast to channel based microfluidics, DMF utilizes alternating currents on an electrode array for moving fluid on the surface of the array. Liquids can thus be moved on an open-plan device by electrowetting. Digital microfluidics allows precise control over the droplet movements including droplet fusion and separation.

Cell-free protein synthesis, also known as in vitro protein synthesis or CFPS, is the production of peptides or proteins using biological machinery in a cell-free system, that is, without the use of living cells. The in vitro protein synthesis environment is not constrained within a cell wall or limited by conditions necessary to maintain cell viability, and enables the rapid production of any desired protein from a nucleic acid template, usually plasmid DNA or RNA from an in vitro transcription. CFPS has been known for decades, and many commercial systems are available. Cell-free protein synthesis encompasses systems based on crude lysate (Cold Spring Harb Perspect Biol. 2016 December; 8(12): a023853) and systems based on reconstituted, purified molecular reagents, such as the PURE system for protein production (Methods Mol Biol. 2014; 1118:275-284). CFPS requires significant concentrations of biomacromolecules, including DNA, RNA, proteins, polysaccharides, molecular crowding agents, and more (Febs Letters 2013, 2, 58, 261-268).

Lab Chip, 2012, 12, 882 (entitled A completely in vitro ultrahigh-throughput droplet-based microfluidic screening system for protein engineering and directed evolution) describes a system based on droplets in a flowing capillary channel, not a three-dimensional array based EWoD system.

U.S. patent application Ser. No. 20/210,016283 describes microwell array systems for high throughput protein expression.

UA20160230203A1 entitled a Portable Fluidic Platform For Rapid Cell-Free Production of Protein Biologics describes an integrated fluidic platform encompassing, cell-free protein synthesis systems coupled to rapid protein purification and characterization modules enabling production of protein biologics.

U.S. Pat. No. 10,464,067 describes Air-matrix digital microfluidics (DMF) apparatuses and methods of using them to prevent or limit evaporation and surface fouling of the DMF apparatus.

WO2004002627 disclose formation of droplets in channels. The droplets are suspended in a continuous phase of silicone oil containing surfactant.

US20070242105, WO2013006312 and WO2017037078 each disclose filler fluids for droplet handling operations in fluidic systems. The filler fluids can contain surfactants.

To date, digital microfluidics, electrowetting-on-dielectric (EWoD), and electrokinesis in general have only found limited uses in cell-free biological-based applications, mostly due to biofouling, where biological components such as proteins, nucleic acids, crude cell extracts and other bioproducts adsorb and/or denature to hydrophobic surfaces. Biofouling is well known in the art to limit the ability of EWoD devices to manipulate droplets containing biomacromolecules. Wheeler and colleagues report that the maximum actuation time for droplets on EWoD devices containing biological media is 30 min before biofouling inhibits EWoD-based droplet actuation (Langmuir 2011, 27, 13, 8586-8594).

Digital microfluidics can be carried out in an air-filled system where the liquid drops are manipulated on the surface in air. However, at elevated temperatures or over prolonged periods, the volatile aqueous droplets simply dry onto the surface by evaporation. This issue is compounded by the high surface area to volume ratio of nanoliter and microliter sized drops. Hence air-filled systems are generally not suitable for protein expression where the temperature of the system needs to be maintained at a temperature suitable for enzyme activity and the duration of the synthesis needs to be prolonged for synthesized proteins levels to be detectable.

### SUMMARY

Here, we report the surprising discovery that adding surfactant to both the oil layer and the aqueous layer gives dramatically improved performance in a digital microfluidic device for use in biomolecule based applications when compared to surfactant in either the aqueous or oil layers alone.

Previous reports have shown that use of surfactants in the oil layer is deleterious. For example, ACS Appl. Mater. Interfaces 2019, 11, 28487-28498 (https://pubs.acs.org/doi/pdf/10.1021/acsami.9b07983) is a demonstration of employing Span80 in dodecane to change contact angle for EWoD. However, when the authors used the composition in a digital microfluidic system (the commercially available OpenDrop), Span80 in dodecane was deleterious and abandoned. Excerpt from Tohgha et al (bold for emphasis):
The manual control system was subsequently used to investigate the minimum voltage required to move nanofluid droplets across the platform. QD-G-COOH drops required a minimum of 200 V to achieve efficient movement without splitting or pinning when pure dodecane oil was employed as the ambient fluid. However, when a droplet of QD-G-COOH was placed in a system of surfactant doped dodecane (1 wt. % Span 80), both the velocity and stability (incomplete droplet transfer) drastically decreased regardless of the voltage applied (120-250 V). Droplets moved slowly between electrodes, often with a visible lag in fluid transport, and the velocity was never greater than ca. 5 mm/s (Movie S2). As noted above, the lower interfacial surface tension that arises from the added surfactant decreases the required actuation voltages for sessile droplets. While EWOD and DMF are similar in action, the driving force for DMF is more closely related to the sum of the electrostatic forces not just the total change in contact angle. Since the decrease in surface tension lowers the total forces on the droplet, there is less work available to efficiently move the droplets along the electrodes. Consequently, all remaining tests on the OpenDrop device were performed in the absence of surfactant.

Assays in the reference above did not use biomolecules such as proteins or nucleic acids. In many instances, the presence of certain surfactants in the aqueous droplets is deleterious to biomolecule interactions.

The inventors have appreciated that the complete removal of surfactants from the aqueous droplets affects the ability to reliably handle droplets on the device. The inventors herein have therefore identified preferred surfactants for use in the aqueous layer in conjunction with preferred surfactants for use in the oil layer. Compositions which contain two or more surfactants are disclosed.

Disclosed is a digital microfluidic device comprising a two-dimensional array of planar electrodes wherein the device comprises a population of aqueous droplets containing biomolecules and a first surfactant within a bulk oil phase, wherein the bulk oil phase contains a second surfactant. The first surfactant may be a non-ionic surfactant, such as for example a pluronic surfactant such as Pluronic F127.

Rather than adding high levels of surfactants to the aqueous biomolecule sample, the applicants have appreciated it is instead beneficial to add an additional surfactant, such as Span85, to the oil. This has the advantages of enabling reactions, including CFPS, to proceed on-DMF without reagents undergoing dilution or adulteration. Additionally, it simplifies the sample preparation procedure for setting up the reactions, increasing the ease of use and the consistency of results.

Using surfactants such as Span85 in the oil layer, such as dodecane, allows for dilution-free CFPS reactions on-DMF, as well as dilution-free detection of the expressed non-fluorescent proteins. Other surfactants besides Span85, and oils other than dodecane could be used. A range of concentrations of Span85 could be used.

Surfactants could be nonionic, anionic, cationic, amphoteric. Each of the surfactants can be mixtures of different surfactants.

Oils could be mineral oils or synthetic oils, including silicone oils, petroleum oils, and perfluorinated oils or a mix thereof.

High levels of surfactants in the aqueous layer can have a detrimental effect on (1) the CFPS reactions and (2) the efficiency of the detection system (if the detection system involves complementation of a tag and detector). Hence this invention where the surfactant is reduced in the aqueous layer has the potential to solve many problems in the use of digital microfluidic (DMF) devices for biological applications. For example, by performing the CFPS reaction on-DMF with oil-surfactant mix, the detection of the expressed protein can proceed without dilution and without needing high levels of aqueous surfactant. It has been shown that high levels of certain surfactants reduce the efficiency of some detection systems reliant on protein-protein interactions, so reducing surfactants from the reagent mix and instead adding them to the oil is beneficial.

Since moving the surfactant to the oil has a benefit of speed and ease of sample handling (such as loading samples onto a digital microfluidic device), this invention could be used and be beneficial for biological reactions which would be performed on-DMF, for example enzymatic DNA synthesis, DNA assembly, protein expression, protein purification, protein binding, and protein activity assays.

The aqueous droplets may contain substantially reduced surfactant compared to systems that operate without surfactant in the oil phase. A minimal level of surfactant in the aqueous droplets may reduce biofouling in certain cases, which along with a minimal level of surfactant in the filler fluid allows both droplet handling and biochemical processes.

The oil can be mineral oil, silicone oil, an alkyl-based solvent, or a fluorinated oil or a blend thereof. The alkyl solvent can be decane or dodecane.

The second surfactant can be a non-ionic surfactant. The second surfactant can be a sorbitan ester. The surfactant can be a Span surfactant. The surfactant can be Span85.

The surfactants can be mixtures of different surfactants. One of the surfactants in the mixture can be a non-ionic surfactant. One of the surfactants can be a sorbitan ester. One of the surfactants can be a Span surfactant. One of the surfactants can be Span85.

The biomolecules can be nucleic acids, for example double stranded nucleic acids. The biomolecules can be peptides. The biomolecules can be proteins.

The droplets can be dispensed, moved, split or combined using a subset of the electrodes on the device.

Disclosed is a method for the cell-free expression of peptides or proteins in a digital microfluidic device wherein the method comprises one or more droplets containing a nucleic acid template and a cell-free system having components for protein expression in an oil-filled environment, wherein the oil contains a surfactant, and moving said droplets using electrowetting-on-dielectric (EWoD).

The cell-free system can be a cell-free extract for protein expression. The cell-free system can be prepared from individual reagents. The method can merge droplets. For example merging a first droplet containing a nucleic acid plasmid with a second droplet containing a cell-free system having the components for protein expression to form a combined droplet on the microfluidic device. The method can be performed on a plurality of droplets having a different nucleic acid template. For example merging a plurality of first droplets containing a nucleic acid template with a plurality of second droplets containing a cell-free system having the components for protein expression to form multiple combined droplets capable of cell-free protein synthesis.

The method can split droplets. The split droplets can be further merged, for example with additive droplets for screening. The droplets can be analysed, for example using optical means such as fluorescence or luminescence. For example, expressed peptides or proteins can be detected by optical means.

Disclosed is a method for the cell-free expression of peptides or proteins in a digital microfluidic device having an oil-filled environment comprising a surfactant, the method comprising:
a. taking a plurality of droplets having a different nucleic acid template,
b. taking a plurality of droplets each containing a cell-free system having the components for protein expression,
c. combining the droplets of a. and b. using electrowetting-on-dielectric phenomena to produce a plurality of droplets capable of expressing proteins of different sequence,
d. mixing the droplets to enable cell-free protein expression, and
e. detecting the expression of proteins within individual droplets.

Disclosed is a method wherein the droplets capable of expressing proteins of different sequence are merged with additive droplets for screening protein expression levels.

Disclosed is a kit for preparing a plurality of peptide or proteins comprising
a. a digital microfluidic device;
b. a reagent source to generate a plurality of droplets containing a cell-free system having the components for protein expression; and
c. an oil, optionally mineral oil, silicone oil, an alkyl-based solvent such as decane or dodecane or a fluorinated oil or a mix thereof, wherein the oil contains a surfactant.

Disclosed is a kit for preparing a plurality of peptide or proteins comprising
a. a digital microfluidic device;
b. a reagent source to generate a plurality of droplets containing a cell-free system having the components for protein expression and a first surfactant; and
c. an oil, optionally mineral oil, silicone oil, an alkyl-based solvent such as decane or dodecane or a fluorinated oil or a mix thereof, wherein the oil contains a second surfactant.

The kit can further include ribosomes, enzymes, initiation factors, nucleotide monomers, amino acid monomers, metal ions and energy sources. In kits the surfactant can be non-ionic, for example Span85.

Disclosed is a kit having reagent droplets containing Pluronic F127 in an oil containing Span85. The oil may be octamethylcyclotetrasiloxane (CTS), decamethyltetrasiloxane (DMTS) or dodecamethylpentasiloxane (DMPS).

Disclosed herein is a method for the monitoring of cell free protein synthesis in a droplet on a digital microfluidic device comprising
a. cell free transcription and translation of a protein of interest fused to a peptide tag; and
b. monitoring the presence of the peptide tag using a further polypeptide which in the presence of the peptide tag produces a detectable signal.

The use of the terms “in vitro” and “cell free” may be used interchangeably herein.

The detectable signal may be for example fluorescence or luminescence. The detectable signal may also be caused by the binding of a ligand to the complemented oligopeptide, peptide, or polypeptide tag fused to the protein of interest. The detectable signal may also be caused by the binding of the polypeptide detector to the protein of interest fused to a His-tag.

Any in vitro transcription and translation may be used, for example extract-based systems derived from rabbit reticulocyte lysate, Chinese Hamster Ovary lysate, a wheat germ, HEK293 lysate, E. coli lysate, yeast lysate.

Alternatively the in vitro transcription and translation may be assembled from purified components, for example a system of purified recombinant elements (PURE).

The in vitro transcription and translation may be coupled or uncoupled.

The peptide tag may be one component of a fluorescent protein and the further polypeptide a complementary portion of the fluorescent protein. The fluorescent protein could include sfGFP, GFP, eGFP, ccGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry₁₁ and the further polypeptide sfCherry₁₋₁₀. The peptide tag may be CFAST₁₁ or CFAST₁₀ and the further polypeptide NFAST in the presence of a hydroxybenzylidene rhodanine analog.

For example, the GFP₁₋₁₀ polypeptide amino acid sequence could be derived from sfGFP:

MSKGEELFTGVVPILVELDGDVNGHKFSVRGEGEGDATNGKLTLKFICT
TGKLPVPWPTLVTTLTYGVQCFSRYPDHMKRHDFFKSAMPEGYVQERTI
SFKDDGTYKTRAEVKFEGDTLVNRIELKGIDFKEDGNILGHKLEYNFNS
HNVYITADKQKNGIKANFKIRHNVEDGSVQLADHYQQNTPIGDGPVLLP
DNHYLSTQSVLSKDPNEK

Alternatively, the GFP₁₋₁₀ polypeptide amino acid sequence could be further mutated from the sequence above to become brighter more quickly upon complementation:

MSKGEELFTGVVPILVELDGDVNGHKFSVRGEGEGDATIGKLTLKFICT
TGKLPVPWPTLVTTLTYGVQCFSRYPDHMKRHDFFKSAMPEGYVQERTI
SFKDDGKYKTRAVVKFEGDTLVNRIELKGTDFKEDGNILGHKLEYNFNS
HNVYITADKQKNGIKANFTVRHNVEDGSVQLADHYQQNTPIGDGPVLLP
DNHYLSTQTVLSKDPNEK

Alternatively, the GFP₁₋₁₀ polypeptide amino acid sequence could be further mutated from the sequence above to have improved properties such as higher solubility or improved expression.

The complementary GFP₁₁ peptide amino acid sequence could be the following:

1.
KRDHMVLLEFVTAAGITGT
2.
KRDHMVLHEFVTAAGITGT
3.
KRDHMVLHESVNAAGIT
4.
RDHMVLHEYVNAAGIT
5.
GDAVQIQEHAVAKYFTV
6.
GDTVQLQEHAVAKYFTV
7.
GETIQLQEHAVAKYFTE

GFP₁₁ or GFP₁₋₁₀ can be fused to the protein of interest through an amino acid linker. In one embodiment, the oligopeptide, peptide, or polypeptide linker can be 0-50 amino acids.

For example, the sfCherry₁₋₁₀ polypeptide amino acid sequence could be:

MEEDNMAIIKEFMRFKVHMEGSVNGHEFEIEGEGEGHPYEGTQTAKLKV
TKGGPLPFAWDILSPQFMYGSKAYVKHPADIPDYLKLSFPEGFTWERVM
NFEDGGVVTVTQDSSLODGEFIYKVKLLGTNFPSDGPVMQKKTMGWEAS
TERMYPEDGALKGEINQRLKLKDGGHYDAEVKTTYKAKKPVQLPGAYNV
DIKLDITSHNED

The complementary sfCherry₁₁ peptide amino acid sequence could be:

YTIVEQYERAEGRHSTGG

sfCherry₁₁ or sfCherry₁₋₁₀ can be fused to the protein of interest through an amino acid linker. In one embodiment, the oligopeptide, peptide, or polypeptide linker can be 0-50 amino acids.

For example, the NFAST polypeptide amino acid sequence could be:

MEHVAFGSEDIENTLAKMDDGOLDGLAFGAIQLDGDGNILQYNAAEGDI
TGRDPKQVIGKNFFKDVAPGTDSPEFYGKFKEGVASGNLNTMFEWMIPT
SRGPTKVKVHMKKALS

The complementary CFAST₁₁ peptide amino acid sequence could be:

GDSYWVFVKRV

Or the complementary CFASTto peptide amino acid sequence could be:

GDSYWVFVKR

NFAST, CFAST₁₁, and/or CFAST₁₀ can be fused to the protein of interest through an amino acid linker. In one embodiment, the oligopeptide, peptide, or polypeptide linker can be 0-50 amino acids.

The peptide tag may also be one component of a protein that forms a detectable substrate, such as a luminescent or colorigenic substrate. The protein could include beta-galactosidase, beta-lactamase, or luciferase.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry₁₁ peptide tags and the synthesis occurs in the presence of multiple sfCherry₁₋₁₀ polypeptides. The protein of interest may be fused to one or more sfCherry₁₁ peptide tags and one or more GFP₁₁ peptide tags and the synthesis occurs in the presence of one or more GFP₁₋₁₀ polypeptides and one or more sfCherry₁₋₁₀ polypeptides.

Any protein of interest may be synthesised. The protein may be an enzyme, for example a terminal deoxynucleotidyl transferase (TdT) enzyme or a truncated version thereof or the homologous amino acid sequence of a terminal deoxynucleotidyl transferase (TdT) enzyme in other species or the homologous amino acid sequence of Polu, PolB, Polk, and Pole of any species or the homologous amino acid sequence of X family polymerases of any species.

The synthesis may be performed in a microfluidic device, for example an electrowetting-on-dielectric (EWoD) device.

### BRIEF DESCRIPTION OF THE DRAWINGS

FIG. 1 shows the dispense success per droplet size (in pixel dimensions) when Span85 in dodecane is used as the oil phase. 1% w/w Span85 in dodecane was the best performing concentration for dispensing small CFPS droplets.

FIG. 2 shows representative images used to generate data in FIG. 1. 46 μl of Span85 (Sigma, #57135-250ML) were added to 5 ml of dodecane (Sigma, #297879-100ML) to constitute the oil phase (1% w/w Span85 in dodecane). (A) Dispense instructions from the software to generate droplets ranging from 4×4 to 8×8 from a series of reservoirs. 4×4 and 6×6 droplets show 100% dispense success, while 8×8 droplets obtain a 11% dispense success. The central right-hand side reservoir was left empty. (B) CFPS droplet dispensed on 1% w/w Span85 in dodecane. (C) Undiluted CFPS dispensed on DMF retains full functionality as demonstrated by the intense fluorescence of lysate expressing GFP constructs. The central left-hand side was loaded with negative CFPS control lacking a DNA construct, hence the dispensed droplets do not show fluorescence. The image was taken after 10 cycles of dispensing the droplets and 3h incubation at room temperature

FIG. 3 shows dispensing of various aqueous reagents. Success in dispensing the CFPS's companion reagents that are routinely used to perform solubility and expressability tests for different proteins: HNG buffer (50 mM HEPES pH 7.4, 100 mM NaCl, 5% v/v glycerol), GFP 1-10 (1 to 10 mg/ml, in different buffers), proteins fused with GFP₁₁ tag in HNG buffer in 1% Span85 in dodecane.

FIG. 4 shows a comparison of dispense and expressability of sfGFP on-DMF between Tween 20 and Span85 systems. On-DMF, undiluted CFPS droplets in 1% w/w Span85 in dodecane show significantly more reliable and higher expression yields than the surfactant-diluted CFPS reaction droplets in dodecane (i.e. where the aqueous droplets contain Tween 20).

FIG. 5 shows schematically one embodiment of the invention. The cell-free protein synthesis reaction contains a nucleic acid template containing the expression cassette for the gene of interest fused to a detectable tag, which is then expressed into the protein of interest through coupled or uncoupled in vitro transcription and in vitro translation. The protein of interest is thus fused to a detectable peptide tag at the N-or C-termini. The nature of the detectable peptide tag is that it can be complemented with a complementary polypeptide resulting in a protein that is fluorescent.

FIG. 6 shows (A) droplets on a digital microfluidic device containing cell-free protein synthesis lysate. The droplets annotated with a white arrow have been merged with recombinant GFP₁₋₁₀ detector polypeptide (t=0). The top two rows additionally contain a DNA construct for the expression of a protein with a GFP₁₁ peptide tag (solid white arrow). (B) Fluorescence image showing the same droplets as in panel (A) after six hours have elapsed. Only the droplets that contain both the expressed protein with GFP₁₁ peptide tag and recombinant GFP₁₋₁₀ detector polypeptide (i.e. solid arrows) show a significant increase in fluorescence. Drops without DNA construct (hollow arrows) or with no GFP₁₋₁₀ detector (no arrows) are not fluorescent. (C) Fluorescence quantification of drops in panel (B). Only the droplets with lysate, DNA construct, and GFP₁₋₁₀ detector polypeptide show a significant increase in fluorescence, indicating protein expression. The negative controls, i.e. bottom row of drops in (B) contain no DNA construct and so low fluorescence, even in the presence of GFP₁₋₁₀ detector. Numbering of drops in (B) and bars in (C) match.

FIG. 7 Images extracted from a time course experiment whereby droplets of cell free protein synthesis (CFPS) lysate, optionally with a DNA construct for a protein of interest (POI—which here is maltose binding protein (MBP) tagged with a GFP₁₁ peptide) and/or GFP₁₋₁₀ polypeptide, were incubated on a digital microfluidic device for 4 hours and imaged periodically. Only the droplets containing lysate, DNA construct, and GFP₁₋₁₀ polypeptide show a significant increase in fluorescence over the course of the experiment, as seen in the right-hand column of images.

FIG. 8A chart showing the real-time fluorescence increase seen in the droplets present in FIG. 7. Quantification of fluorescence was performed using ‘Image J’ and the values presented have been subject to normalisation by subtracting the background fluorescence seen in droplets of CFPS lysate and GFP₁₋₁₀ (i.e. no DNA construct so no protein of interest, POI, expressed). Only the droplets containing all components—lysate, DNA construct for POI, and GFP₁₋₁₀ polypeptide—generate fluorescence over background.

FIG. 9 In this experiment, the recombinantly purified GFP₁₋₁₀ detector was added to the cell free lysate at the same time as the DNA construct encoding for a GFP₁₁-tagged protein. The fluorescent signal was monitored over time in a plate reader. A chart showing fluorescence signal increases over time from plate reader measurements of CFPS reactions. GFP₁₋₁₀ detector is present in all three reactions from the start, enabling real-time detection of protein expression. In the two conditions which have a protein of interest fused to a GFP₁₁ tag, fluorescence increases compared to the negative control condition where there is no GFP₁₁-tagged protein. POI1 is an engineered terminal transferase and POI2 is SARS-COV-NL63-Mpro. Both had a 3xGFP₁₁ tag at the N terminus.

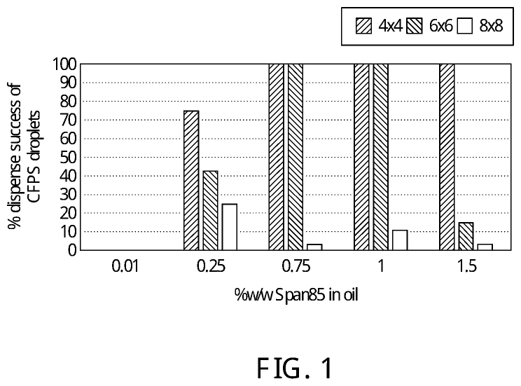

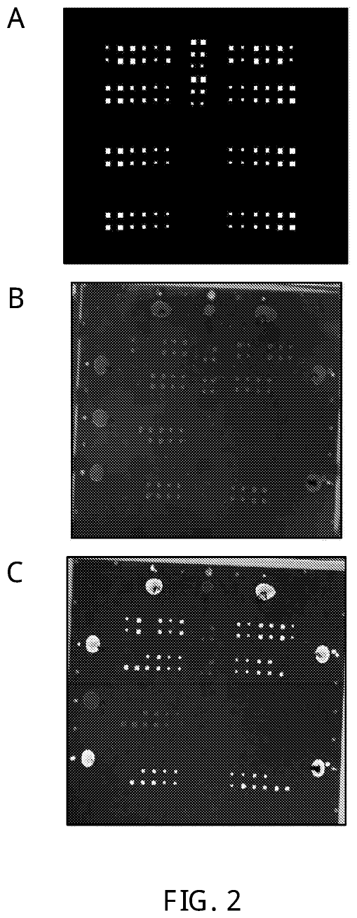

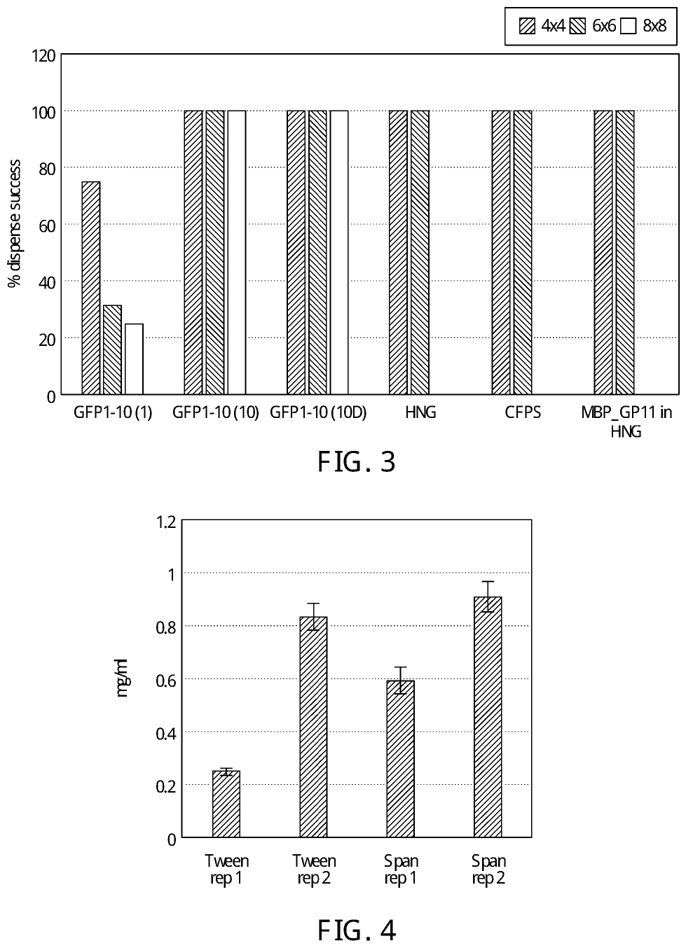

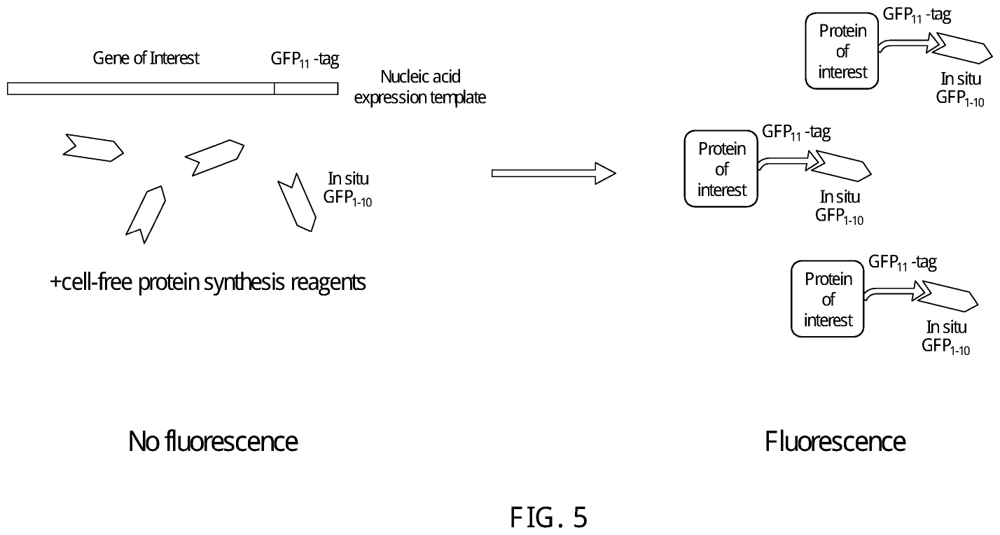

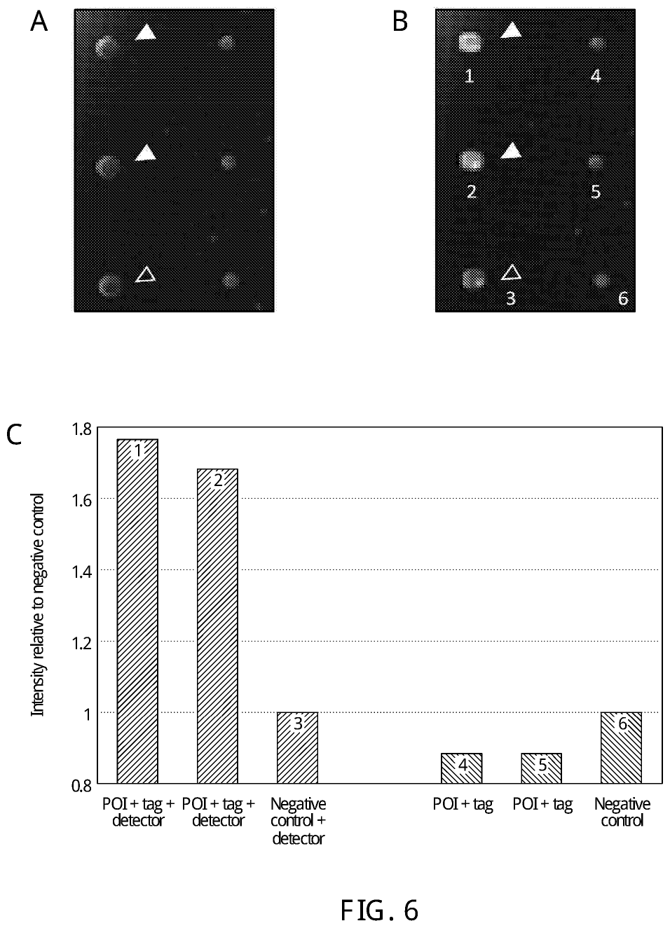

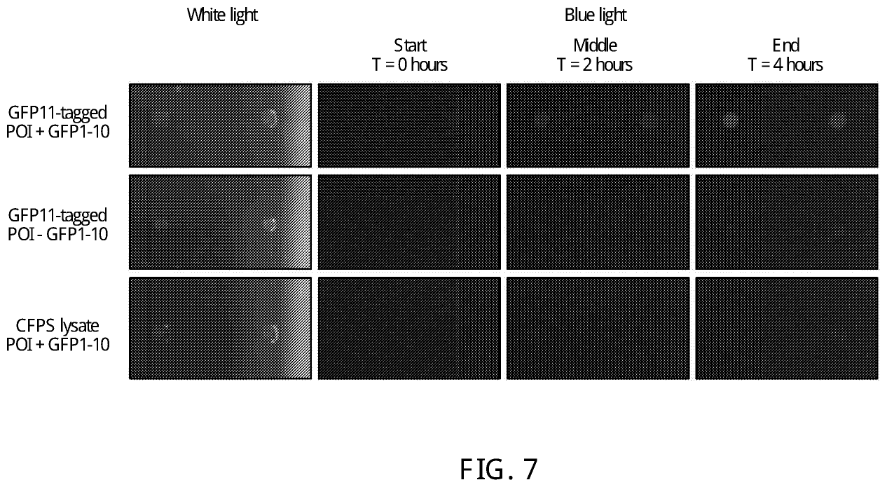

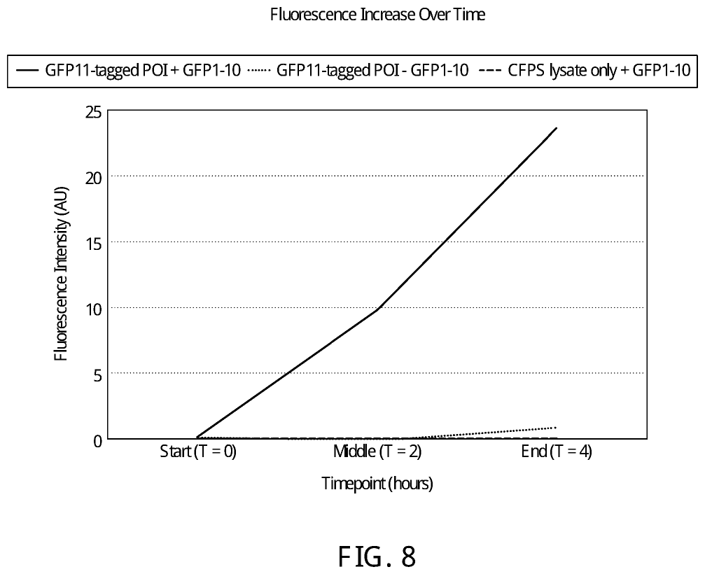

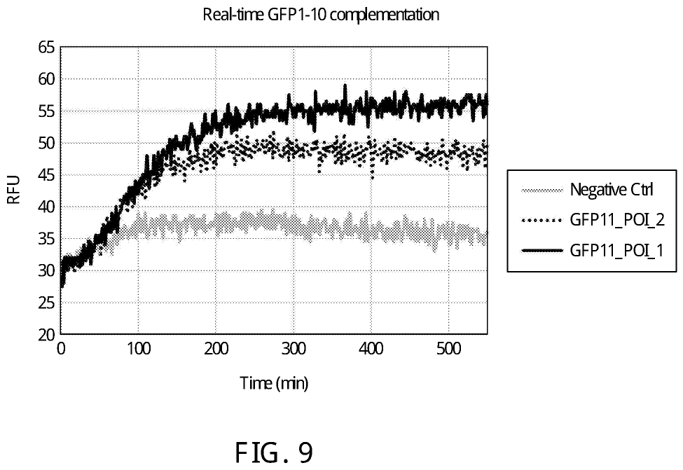

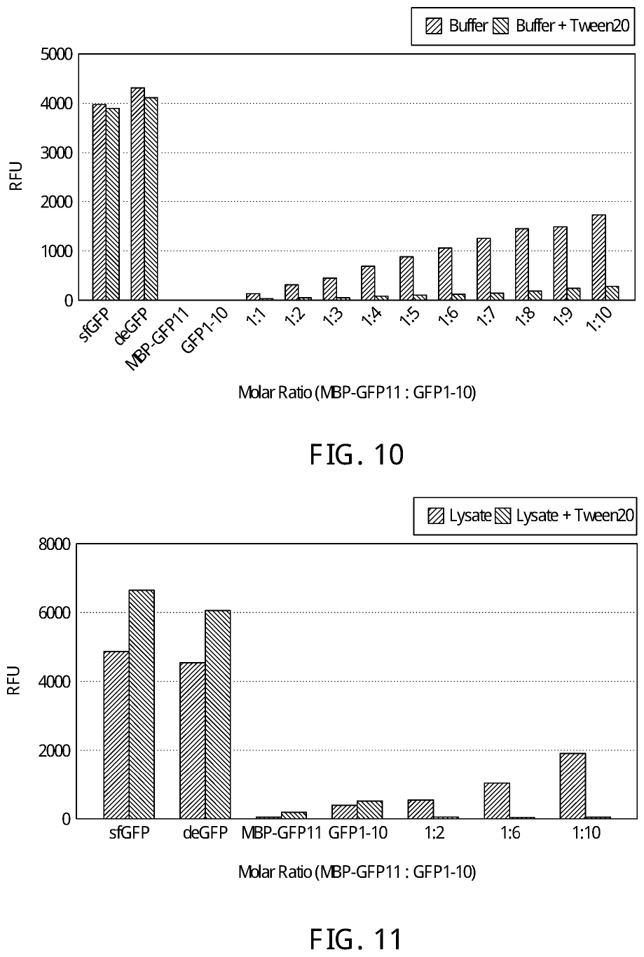

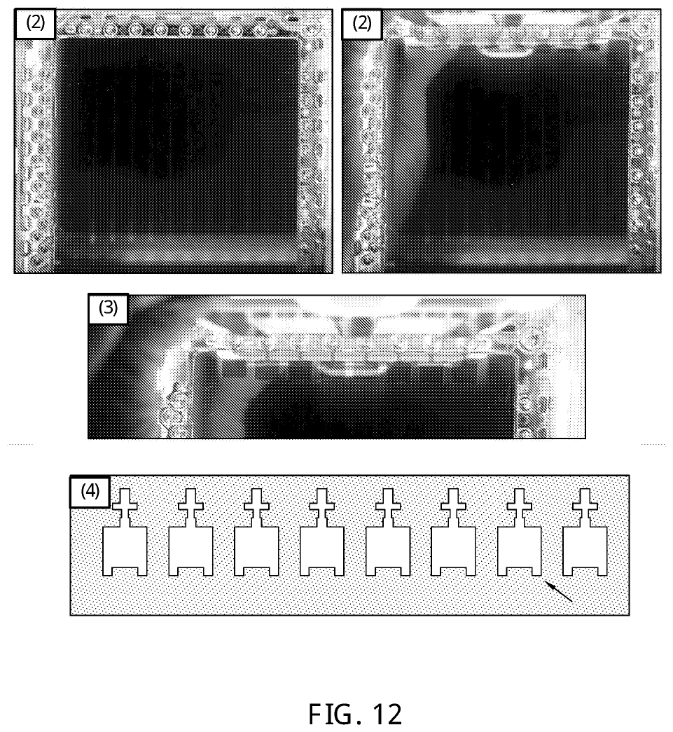

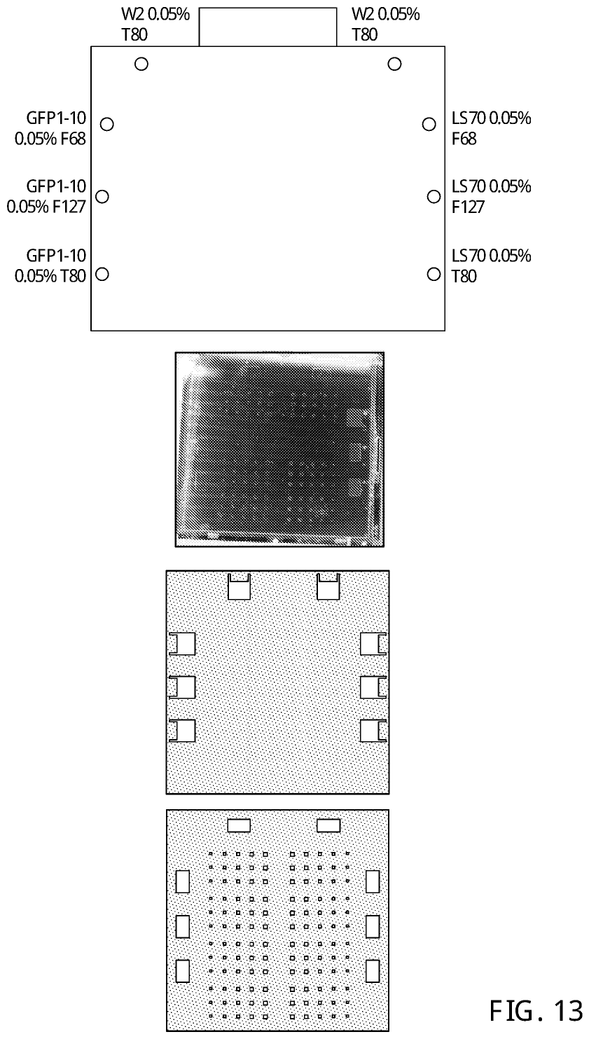

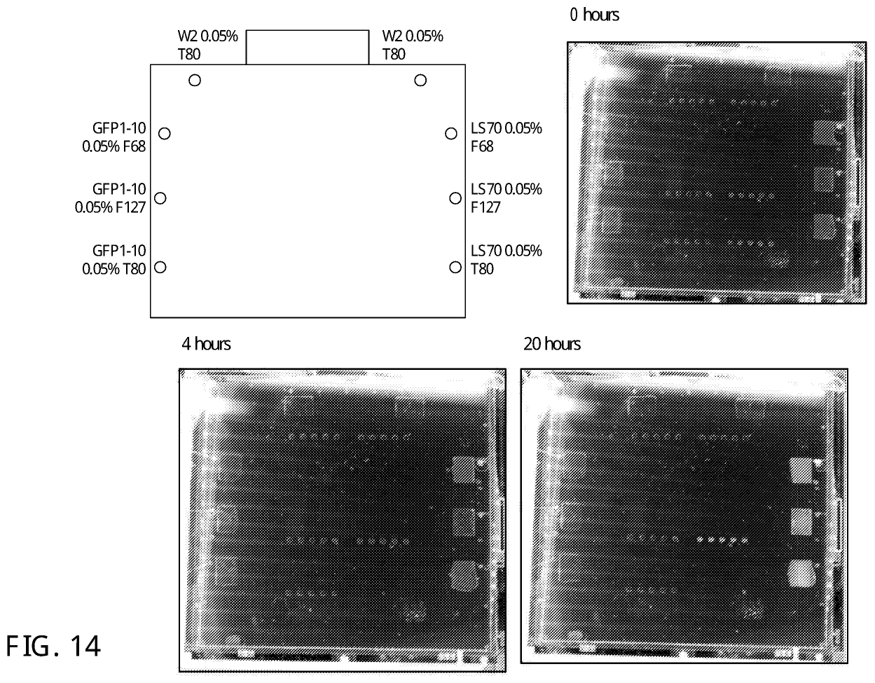

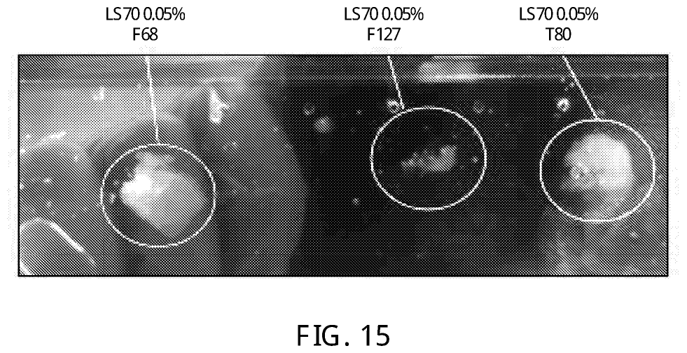

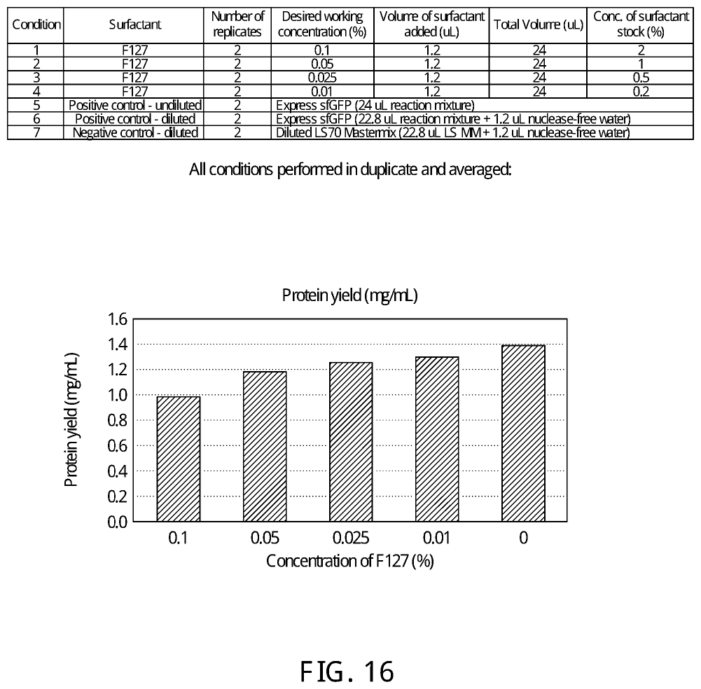

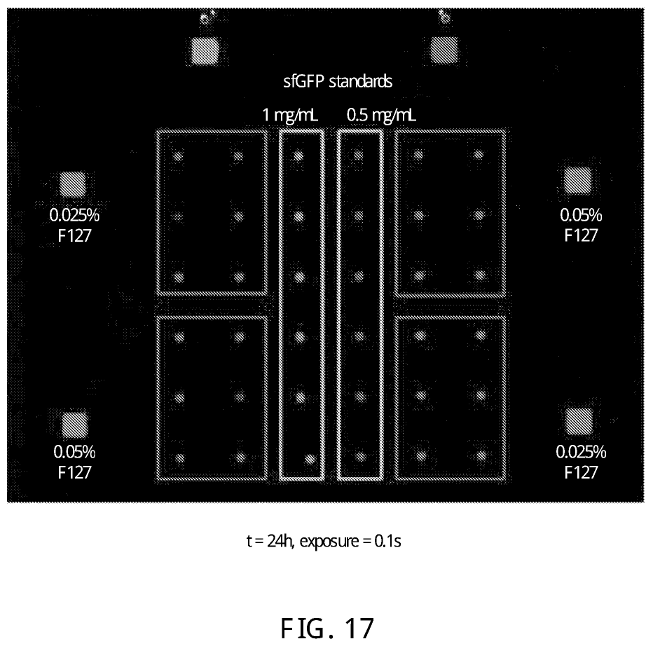

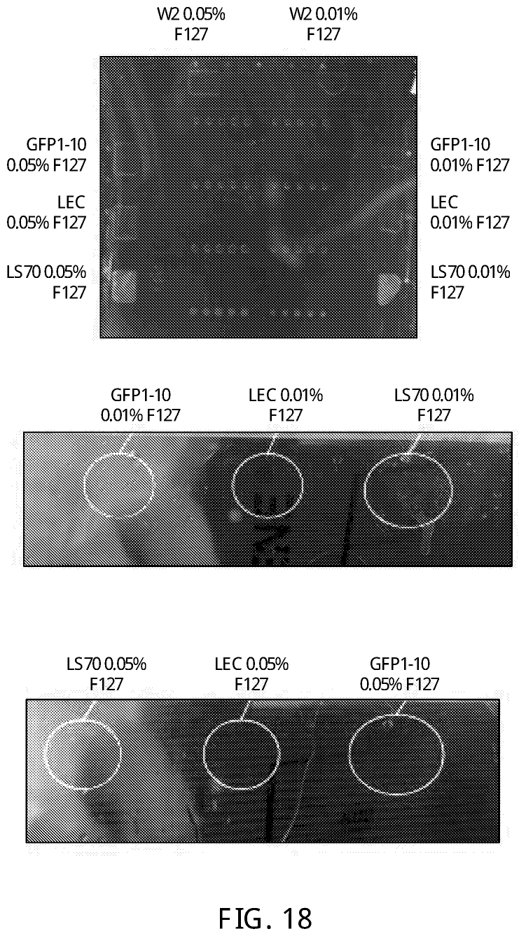

FIGS. 7-9 demonstrate real-time detection of protein expression. GFP₁₋₁₀ detector polypeptide is present right from the start of the experiment. Fluorescent signal increases as GFP₁₁ tags are expressed. These experiments were performed in a base fluid comprising 0.2% Span 85 in dodecamethylpentasiloxane rather than Tween20 in aqueous and no surfactant in dodecane.

FIG. 10 Demonstration that the complementation of the GFP₁₁ tag with recombinant GFP₁₋₁₀ detector is inhibited in the presence of 0.1% w/v Tween20 surfactant. The complementation assay was performed in TNG Buffer (50 mM Tris, pH 7.4, 0.1 M NaCl, 10% v/v glycerol). Fluorescence was measured after 24 hours incubation at 29° C. The first four pairs are controls: sfGFP and deGFP are complete fluorescent proteins, while MBP-GFP₁₁ and GFP₁₋₁₀ are partial fluorescent proteins (tag and detector respectively) hence show no fluorescence. The ten sample pairs have the same quantity of MBP-GFP₁₁ but increasing molar excesses of recombinant GFP₁₋₁₀ detector polypeptide. The data also shows that increasing the molar excess of GFP₁₋₁₀ detector polypeptide over the GFP₁₁ peptide tag leads to enhanced fluorescence signal, and that the presence of Tween significant affects the signal due to lower levels of protein assembly.

FIG. 11 Demonstration that the complementation of the GFP₁₁ tag with recombinant GFP₁₋₁₀ detector is inhibited in the presence of 0.1% w/v Tween20 surfactant. The complementation assay was performed in a cell-free lysate (070 Linear Master Mix, Arbor Bioscience). Fluorescence was measured after 24 hours incubation using a plate reader. The first four pairs are controls: sfGFP and deGFP are complete fluorescent proteins, while MBP-GFP₁₁ and GFP₁₋₁₀ are partial fluorescent proteins (tag and detector respectively) hence show no fluorescence. The three sample pairs have the same quantity of MBP-GFP₁₁ but increasing molar excesses of recombinant GFP₁₋₁₀ detector polypeptide.

FIG. 12 shows a sequence of images (1-3) demonstrating formation of eight aqueous reservoirs with calibration structures, driven with an air displacement multichannel pipette. The arrow shows the formation of calibration structures on the reservoirs. The volume of the aqueous phase loaded is 5 μL, including both the reservoir to be formed and the calibration structures. The sizes of the actuated areas are 30×28 pixels for the main reservoir and 6×6 pixels for each of the calibration structures. The time required to fill the reservoirs was 120 seconds. The aqueous reagent loaded is 0.05% w/w Pluronic F127 in an aqueous buffer with red food colouring to aid visualisation (1:1 dilution). The filler fluid in the device is 0.1% span85 in dodecamethylpentasiloxane (DMPS). Image (4) shows a snapshot of the electrical actuation pattern sent to the electrodes on the device during reservoir filling, where white represents electrodes with a potential applied. The calibration structures are shown by the arrow on the image. FIG. 4 image (1) shows a DMF device primed with filler fluid. FIG. 4 image (2) shows the initial stages of reservoir loading. FIG. 4 image (3) shows two reservoirs filled to the correct volume (both calibration structures visible) while other reservoirs are still in the process of forming on the device.

FIG. 13 shows representative images from an EWoD device that compares the performance of aqueous surfactants: F127, Tween 80, F68 at 0.05% v/v in a base fluid of dodecane with 0.1% span 85 as oil phase. The images indicate F127 is better than F68 and comparable to Tween 80 when dispensing droplets from a reservoir. The first images shows reagent locations. The second shows reagent droplets of varying sizes. The third and fourth images indicate the start and finish pixel driving locations.

FIG. 14 shows a series of dispenses over time. Over long periods of time the F127 is the only detergent allowing suitable dispenses (middle row). Two different aqueous reagents (LS70 and GFP₁₋₁₀) and three detergents were compared with dispenses at 0, 4 and 20 hours. Basefluid dodecane with 0.1% span 85 as oil phase with 3X aqueous surfactants at 0.05% in for comparison. A time zero, both Tween 80 and F127 dispense equally. After 4 and 20 hours, the LS70 in Tween80 fails to dispense, whereas the F127 continues to be operational.

FIG. 15 shows biofouling at the end of the experiment from FIG. 14. F68 and Tween 80 show significant protein biofouling from the aqueous reagent having GFP₁₋₁₀. F127 (central circle) has a lower level of biofouling.

FIG. 16 shows an in-tube screen of the expression of sfGFP with a variation of F127 concentration. Higher levels of detergent inhibit the level of protein expression. The basic conclusion is that detergents are good for droplet manipulation and device handling, but bad for biochemical processes. The concentrations of particular detergents should therefore be optimised.

FIG. 17 shows expression of sfGFP in droplets on an electrowetting device. The data indicated no substantial difference in yield between 0.025% and 0.05% F127. DMPS/0.1% Span85 used as basefluid. Protein yield 0.05% F127: 0.56 mg/ml; 0.025% F127: 0.56 mg/mL. Off-device yield: 0.05%: 0.72 mg/ml; 0.025%: 0.67 mg/ml. Reducing Pluronic F127 concentration in aqueous phase did not result in increased protein yield on the device. Thus the presence of pluronic surfactants, unlike Tween surfactants, does not prevent detection of protein expression.

FIG. 18 shows several dispensed droplets (at time point=20 hrs) of a fixed and same size from three different aqueous reagents (Lysate, LEC (linear expression construct, i.e. DNA) and GFP₁₋₁₀) containing two different concentration of F127; 0.05% and 0.01% v/v. Basefluid is DMPS with 0.1% span 85 as oil. Middle and the bottom image shows biofouling for only 0.01% F127 for LS70 at the end of the experiment.

### DETAILED DESCRIPTION OF THE INVENTION

Disclosed herein is a digital microfluidic device comprising a two-dimensional array of planar electrodes wherein the device comprises a population of aqueous droplets containing biomolecules within a bulk oil phase, wherein the bulk oil phase contains a surfactant. The inventors have appreciated that combinations of two or more surfactants are beneficial, one in the aqueous layer and one in the oil layer.

Disclosed herein is a method for the monitoring of cell free protein synthesis in a droplet on a digital microfluidic device comprising
a. cell free transcription and translation of a protein of interest fused to a peptide tag; and
b. monitoring the presence of the peptide tag using a further polypeptide which in the presence of the peptide tag produces a detectable signal.

Disclosed is a method for the cell-free expression of peptides or proteins in a digital microfluidic device. The droplets having the components required for cell-free protein synthesis (CFPS), otherwise known as in vitro protein synthesis, can be manipulated by electrokinesis in order to effect and improve protein expression. The droplets are generally lacking in surfactant beyond surfactant equilibrated from the oil layer.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrokinesis. The components for the cell-free protein synthesis droplet can be pre-mixed prior to introduction to or mixed on the digital microfluidic device. The oil layer contains the surfactant required on the device.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static.

The droplet can be moved using any means of electrokinesis. The droplet can be moved using electrowetting-on-dielectric (EWoD). The electrical signal on the EWoD or optical EWoD device can be delivered through segmented electrodes, active-matrix thin-film transistors, or digital micromirrors.

The oil in the device can be any water immiscible liquid. The oil can be mineral oil, silicone oil such as dodecamethylpentasiloxane, an alkyl-based solvent such as decane or dodecane, or a fluorinated oil. The oil can be oxygenated prior to or during the expression process.

The silicone oil can be octamethylcyclotetrasiloxane (CTS), decamethyltetrasiloxane (DMTS) or dodecamethylpentasiloxane (DMPS).

The surfactant in the aqueous layer can be a pluronic surfactant. Pluronic surfactants are also known as poloxamers, and are a class of synthetic block copolymers which consist of hydrophilic poly (ethylene oxide) (PEO) and hydrophobic poly (propylene oxide) (PPO), arranged in an A-B-A triblock structure, thus giving PEO-PPO-PEO. The surfactant may be Pluronic F127.

The pluronic surfactant can be present at less than 0.1%. High levels of surfactant are detrimental to the detection of protein expression. The pluronic concentration can be between 0.025 and 0.1%. The concentration may be 0.05%.

Disclosed is a composition comprising 0.05% w/w Pluronic F127 in an aqueous buffer in a filler fluid of 0.1% span85 in dodecamethylpentasiloxane (DMPS) and use thereof in electrowetting applications, including protein expression.

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with additive droplets for screening.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

The term digital microfluidic device refers to a device having a two-dimensional array of planar microelectrodes. The term excludes any devices simply having droplets in a flow of oil in a channel. The droplets are moved over the surface by electrokinetic forces by activation of particular electrodes. Upon activation of the electrodes the dielectric layer becomes less hydrophobic, thus causing the droplet to spread onto the surface. A digital microfluidic (DMF) device set-up is known in the art, and depends on the substrates used, the electrodes, the configuration of those electrodes, the use of a dielectric material, the thickness of that dielectric material, the hydrophobic layers, and the applied voltage.

Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk oil.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

Digital microfluidics (DMF) refers to a two-dimensional planar surface platform for lab-on-a-chip systems that is based upon the manipulation of microdroplets. Droplets can be dispensed, moved, stored, mixed, reacted, or analyzed on a platform with a set of insulated electrodes. Digital microfluidics can be used together with analytical analysis procedures such as mass spectrometry, colorimetry, electrochemical, and electrochemiluminescense.

The droplet can be moved using any means of electrokinesis. The aqueous droplet can be moved using electrowetting-on-dielectric (EWoD). Electrowetting on a dielectric (EWoD) is a variant of the electrowetting phenomenon that is based on dielectric materials. During EWoD, a droplet of a conducting liquid is placed on a dielectric layer with insulating and hydrophobic properties. Upon activation of the electrodes the dielectric layer becomes less hydrophobic, thus causing the droplet to spread onto the surface.

The electrical signal on the EWoD or optically-activated amorphous silicon (a-Si) EWoD device can be delivered through segmented electrodes, active-matrix thin-film transistors or digital micromirrors. Optically-activated s-Si EWoD devices are well known in the art for actuating droplets (J. Adhes. Sci. Technol., 2012, 26, 1747-1771).

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be split on the device either before, during or after expression. Included herein is a method further comprising splitting the droplet into multiple droplets. If desired the split droplets can be screened with further additives. Included is a method wherein one of more of the split droplets are merged with additive droplets for screening.

Through an affinity tag, such as a FLAG-tag, HIS-tag, GST-tag, MBP-tag, STREP-tag, or other form of affinity tag, CFPS-expressed proteins can be immobilized to a solid-support affinity resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF-and CE-CFPS, users can scale up their CFPS production methods.

The droplets can be actuated on a hydrophobic surface on the digital microfluidic device (ACS Nano 2018, 12, 6, 6050-6058). The hydrophobic surface can be a hydrophobic surface such as polytetrafluoroethylene (PTFE), Teflon AF (DuPont Inc), CYTOP (AGC Chemicals Inc), or FluoroPel (Cytonix LLC). The hydrophobic surface may be modified in such a way to reduce biofouling, especially biofouling resulting from exposure to CFPS reagents or nucleic acid reagents. The hydrophobic surface may also be superhydrophobic, such as NeverWet (NeverWet LLC) or Ultra-Ever Dry (Flotech Performance Systems Ltd). Superhydrophobic surfaces prevent biofouling compared with typical fluorocarbon-based hydrophobic surfaces. Superhydrophobic surfaces thus prolong the capability of digital microfluidic devices to move CFPS droplets and general solutions containing biopolymers (RSC Adv., 2017, 7, 49633-49648). The hydrophobic surface can also be a slippery liquid infused porous surface (SLIPS), which can be formed by infusing Krtox-103 oil (DuPont) with porous PTFE film (Lab Chip, 2019, 19, 2275).

For electrowetting on dielectrics (EWoD), the change in contact angle of reagent upon the application of electric potential is an inverse function of surface tension. Thus, for low voltage EWoD operations, reduction in surface tension is achieved by addition of surfactants to reagents, which for CFPS reactions means to the lysate and to the DNA. This results in a dilution of the lysate, and it has been seen, in experiments, that diluting the lysate results in a decrease in expression level of the protein of interest. Thus performing CFPS on DMF where the surfactants are added to the solutions being moved will necessarily result in a dilution of the lysate and thus a decrease in the level of protein expression. In addition to being a problem in its own right, this further complicates extrapolation of on-DMF results to in-tube predictions of protein yield. An additional detriment of having to add surfactants to the samples is that this increases the time required for sample preparation, as well as increasing the potential for inconsistent results due to ‘user error,’ as there is more handling of reagents. An additional detriment of having to add surfactants to the samples is that certain downstream operations are hindered. For example, if a protein of interest is expressed in a cell free system with a GFP₁₁ (or similar) peptide tag, it's downstream complementation with a GFP₁₋₁₀ detector polypeptide is hindered in the presence of surfactant.

Rather than adding surfactants to the aqueous sample, it is instead possible to add surfactant, such as Span85 (e.g. https://www.sigmaaldrich.com/GB/en/product/mm/840124), to the oil. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution and without adding aqueous surfactant. It has been shown that surfactants reduce the efficiency of some detection systems, including but not limited to the Split GFP system, so removing surfactants from the reagent mix and instead adding them to the oil can be beneficial.

The filler fluid may be selected to have a particular surface or interfacial tension with the droplet phase or with the droplet microactuator surfaces. Surfactants can be added to the filler fluid to stabilize liquid films that may be present between the droplet and solid phases. Examples of suitable surfactants include nonionic low HLB (hydrophile-lipophile balanced) surfactant. The HLB preferably less than about 10 or less than about 5. Suitable examples include: Triton X-15 (HLB=4.9) (octylphenol ethoxylate); Span 85 (HLB 1.8) (sorbitan trioleate); Span 65 (2.1) (sorbitan tristearate); Span 83 (3.7) (sorbitan sesquioleate); Span 80 (4.3) (sorbitan monoleate); Span 60 (4.7) (sorbitan monostearate); and fluorinated surfactants.

The peptide tag can be attached to the C or N terminus of the protein. The peptide tag may be one component of a green fluorescent protein (GFP). For example the peptide tag may be GFP₁₁ and the further polypeptide GFP₁₋₁₀. The peptide tag may be one component of sfCherry. The peptide tag may be sfCherry11 and the further polypeptide sfCherry₁₋₁₀.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry11 peptide tags and the synthesis occurs in the presence of multiple sfCherry₁₋₁₀ polypeptides. The protein of interest may be fused to one or more sfCherry11 peptide tags and one or more GFP₁₁ peptide tags and the synthesis occurs in the presence of one or more GFP₁₋₁₀ polypeptides and one or more sfCherry₁₋₁₀ polypeptides.

Assays improved by removing surfactants from the aqueous layer may include nucleic acid synthesis, nucleic acid construction or protein/protein interactions.

### Devices

The manipulation of droplets by the application of electrical potential can be achieved on electrodes covered with an insulator or a dielectric or a series of insulators or dielectrics. Droplet manipulation as a result of an applied electrical potential is known as electrowetting. Electrokinesis occurs as result of a non-uniform electric field that influences the hydrostatic equilibrium of a dielectric liquid (dielectrophoresis or DEP) or a change in the contact angle of the liquid on solid surface (electrowetting-on-dielectric or EWoD). DEP can also be used to create forces on polarizable particles to induce their movement. The electrical signal can be transmitted to a discrete electrode, a transistor, an array of transistors, or a sheet of semi-conductor film whose electrical properties can be modulated by an optical signal.

EWoD phenomena occur when droplets are actuated between two parallel electrodes covered with a hydrophobic insulator or dielectric. The electric field at the electrode-electrolyte interface induces a change in the surface tension, which results in droplet motion as a result of a change in droplet contact angle. The electrowetting effect can be quantitatively treated using Young-Lippmann equation:

$$cos θ - cos {θ}_{0} = ( 1 / 2 γ LG ) c \cdot {V}^{2}$$

where θ₀ is the contact angle when the electric field across the interfacial layer is zero, γLG is the liquid-gas tension, c is the specific capacitance (given as εᵣ. ε₀/t, where εᵣ is dielectric constant of the insulator/dielectric, ε₀ is permittivity of vacuum, t is thickness) and V is the applied voltage or electrical potential. The change in contact angle (inducing droplet movement) is thus a function of surface tension, electrical potential, dielectric thickness, and dielectric constant.

When a droplet is actuated by EWoD, there are two opposing sets of forces that act upon it: an electrowetting force induced by electric field and resistant forces that include the drag forces resulting from the interaction of the droplet with filler medium and the contact line friction (ref). The minimum voltage applied to balance the electrowetting force with the sum of all drag forces (threshold voltage) is variably determined by the thickness-to-dielectric contact ratio of the insulator/dielectric, (t/εᵣ)^{1/2}. Thus, to reduce actuation voltage, it is required to reduce (t/εᵣ)^{1/2} (i.e., increase dielectric constant or decrease insulator/dielectric thickness). To achieve low voltage actuation, thin insulator/dielectric layers must be used. However, the deposition of high quality thin insulator/dielectric layers is a technical challenge, and these thin layers are easily damaged before the desired electrowetting contact angle is large enough to drive the droplet is achieved. Most academic studies thus report the use of much higher voltages >100V on easily fabricated, thick dielectric films (>3 μm) to effect electrowetting.

High voltage EWoD-based devices with thick dielectric films, however, have limited industrial applicability largely due to their limited droplet multiplexing capability. The use of low voltage devices including thin-film transistors (TFT) and optically-activated amorphous silicon layers (a-Si) have paved the way for the industrial adoption of EWoD-based devices due to their greater flexibility in addressing electrical signals in a highly multiplex fashion. The driving voltage for TFTs or optically-activated a-Si are low (typically <15 V). The bottleneck for fabrication and thus adoption of low voltage devices has been the technical challenge of depositing high quality, thin film insulators/dielectrics. Hence there has been a particular need for improving the fabrication and composition of thin film insulator/dielectric devices.

Typically, the electrodes (or the array elements) used for EWoD are covered with (i) a hydrophilic insulator/dielectric and a hydrophobic coating or (ii) a hydrophobic insulator/dielectric. Commonly used hydrophobic coatings comprise of fluoropolymers such as Teflon AF 1600 or CYTOP. The thickness of this material as a hydrophobic coating on the dielectric is typically <100 nm and can have defects in the form of pinholes or a porous structure; hence, it is particularly important that the insulator/dielectric is pinhole free to avoid electrical shorting. Teflon has also been used as an insulator/dielectric, but it has higher voltage requirements due to its low dielectric constant and the thickness required to make it pinhole free. Other hydrophobic insulator/dielectric materials can include polymer-based dielectrics such as those based on siloxane, epoxy (e.g. SU-8), or parylene (e.g., parylene N, parylene C, parylene D, or parylene HT). Due to minimal contact angle hysteresis and a higher contact angle with aqueous solutions, Teflon is still used as a hydrophobic topcoat on these insulator/dielectric polymers. However, there are difficulties in reliably producing <1 micron pinhole-free coatings of parylene or SU-8; thus, the thickness of these materials is typically kept at a 2-5 microns at the cost of increased voltage requirements for electrowetting. It has also been reported that traditional EWoD devices with parylene C are easily broken and unstable for repeated droplet manipulation with cell culture medium. Multi-layer insulator devices deposited with metal-oxide and parylene C films have been used to produce a more robust insulator/dielectric and enable operations with lower applied voltages. Inorganic materials, such metal oxides and semiconductor oxides, commonly used in the CMOS industry as “gate dielectrics”, have been used as insulator/dielectric for EWoD devices. They offer the advantage of utilizing standard cleanroom processes for thin film depositions (<100 nm). These materials are inherently hydrophilic, requiring an additional hydrophobic coating, and can be prone to pinhole formation as a result of thin film layer deposition process. Together with the need for lower voltage operations of EWoD, recent developmental work has focused on (1) using materials with improved dielectric properties (e.g., using high-dielectric constant insulators/dielectrics), (2) optimizing the fabrication process to make the insulator/dielectric pinhole free to avoid dielectric breakdown.

Operation of EWoD devices suffers from contact angle saturation and hysteresis, which is believed to be brought about by either one or combination of these phenomena: (1) entrapment of charges in the hydrophobic film or insulator/dielectric interface, (2) adsorption of ions, (3) thermodynamic contact angle instabilities, (4) dielectric breakdown of dielectric layer, (5) the electrode-electrode-insulator interface capacitance (arising from the double layer effect), and (6) fouling of the surface (such as by biomacromolecules). One of the adverse effects of this hysteresis is reduced operational lifetime of the EWoD-based device.

Contact angle hysteresis is believed to be a result of charge accumulation at the interface or within the hydrophobic insulator after several operations. The required actuation voltage increases due to this charging phenomenon resulting in eventual catastrophic dielectric breakdown. The most probable explanation is that pinholes at the insulator/dielectric may allow the liquid to come into contact with the electrode causing electrolysis. Electrolysis is further facilitated by pinhole-prone or porous hydrophobic insulators.

Most of the studies to understand contact angle hysteresis on EWoD have been conducted on short time scales and with low conductivity solutions. Long duration actuations (e.g., >1 hour) and high conductivity solutions (e.g., 1 M NaCl) could produce several effects other than electrolysis. The ions in solution can permeate through the hydrophobic coat (under the applied electric field) and interact with the underlying insulator/dielectric. Ion permeation can result in (1) change in dielectric constant due to charge entrapment (which is different from interfacial charging) and (2) change in surface potential of a pH sensitive metal oxide. Both can result in reduction of electrowetting forces to manipulate aqueous droplets, leading to contact angle hysteresis. The inventors have previously found that the damage from high conductivity solutions reduces or disables electrowetting on electrodes by inhibiting the modulation of contact angle when an electric field is applied.

An electrokinetic device includes a first substrate having a matrix of electrodes, wherein each of the matrix electrodes is coupled to a thin film transistor, and wherein the matrix electrodes are overcoated with a functional coating comprising: a dielectric layer in contact with the matrix electrodes, a conformal layer in contact with the dielectric layer, and a hydrophobic layer in contact with the conformal layer; a second substrate comprising a top electrode; a spacer disposed between the first substrate and the second substrate and defining an electrokinetic workspace; and a voltage source operatively coupled to the matrix electrodes.

The dielectric layer may comprise silicon dioxide, silicon oxynitride, silicon nitride, hafnium oxide, yttrium oxide, lanthanum oxide, titanium dioxide, aluminum oxide, tantalum oxide, hafnium silicate, zirconium oxide, zirconium silicate, barium titanate, lead zirconate titanate, strontium titanate, or barium strontium titanate. The dielectric layer may be between 10 nm and 100 μm thick. Combinations of more than one material may be used, and the dielectric layer may comprise more than one sublayer that may be of different materials.

The conformal layer may comprise a parylene, a siloxane, or an epoxy. It may be a thin protective parylene coating in between the insulating dielectric and the hydrophobic coating. Typically, parylene is used as a dielectric layer on simple devices. In this invention, the rationale for deposition of parylene is not to improve insulation/dielectric properties such as reduction in pinholes, but rather to act as a conformal layer between the dielectric and hydrophobic layers. The inventors find that parylene, as opposed to other similar insulating coatings of the same thickness such as PDMS (polydimethylsiloxane), prevent contact angle hysteresis caused by high conductivity solutions or solutions deviating from neutral pH for extended hours. The conformal layer may be between 10 nm and 100 μm thick.

The hydrophobic layer may comprise a fluoropolymer coating, fluorinated silane coating, manganese oxide polystyrene nanocomposite, zinc oxide polystyrene nanocomposite, precipitated calcium carbonate, carbon nanotube structure, silica nanocoating, or slippery liquid-infused porous coating.

The elements may comprise one or more of a plurality of array elements, each element containing an element circuit; discrete electrodes; a thin film semiconductor in which the electrical properties can be modulated by incident light; and a thin film photoconductor whose properties can be modulated by incident light.

The functional coating may include a dielectric layer comprising silicon nitride, a conformal layer comprising parylene, and a hydrophobic layer comprising an amorphous fluoropolymer. This has been found to be a particularly advantageous combination.

The electrokinetic device may include a controller to regulate a voltage provided to the individual matrix electrodes. The electrokinetic device may include a plurality of scan lines and a plurality of gate lines, wherein each of the thin film transistors is coupled to a scan line and a gate line, and the plurality of gate lines are operatively connected to the controller. This allows all the individual elements to be individually controlled.

The second substrate may also comprise a second hydrophobic layer disposed on the second electrode. The first and second substrates may be disposed so that the hydrophobic layer and the second hydrophobic layer face each other, thereby defining the electrokinetic workspace between the hydrophobic layers.

The method is particularly suitable for aqueous droplets with a volume of 1 μl or smaller.

The EWoD-based devices shown and described below are active matrix thin film transistor devices containing a thin film dielectric coating with a Teflon hydrophobic top coat. These devices are based on devices described in the E Ink Corp patent filing on “Digital microfluidic devices including dual substrate with thin-film transistors and capacitive sensing”, US patent application no 2019/0111433, incorporated herein by reference.

Described herein are electrokinetic devices, including:
a first substrate having a matrix of electrodes, wherein each of the matrix electrodes is coupled to a thin film transistor, and wherein the matrix electrodes are overcoated with a functional coating comprising:
a dielectric layer in contact with the matrix electrodes,
a conformal layer in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

Described herein is an electrokinetic device, including:
a first substrate having a matrix of electrodes, wherein each of the matrix electrodes is coupled to a thin film transistor, and wherein the matrix electrodes are overcoated with a functional coating comprising:
one or more dielectric layer(s) comprising silicon nitride, hafnium oxide or aluminum oxide in contact with the matrix electrodes,
a conformal layer comprising parylene in contact with the dielectric layer, and
a hydrophobic layer in contact with the conformal layer;
   a second substrate comprising a top electrode;
   a spacer disposed between the first substrate and the second substrate and defining an electrokinetic workspace; and
   a voltage source operatively coupled to the matrix electrodes;

The electrokinetic devices as described may be used with other elements, such as for example devices for heating and cooling the device or reagent cartridges for the introduction of reagents as needed.

### Applications of the Invention

The invention can be used in a myriad of different applications. In particular the invention can be used to move cells, nucleic acids, nucleic acid templates, proteins, initiation oligonucleotide sequences for nucleic acid synthesis, beads, magnetic beads, cells immobilised on magnetic beads, or biopolymers immobilised on magnetic beads.

In these applications the steps of disposing an aqueous droplet having an ionic strength on a first matrix electrode and providing a differential electrical potential may be repeated many times. They may be repeated over 1000 times or over 10,000 times, sometimes over a 24 hour period.

### Nucleic Acid Syntheses Applications

The present method can be used in the synthesis of nucleic acids, such as phosphoramidite-based nucleic acid synthesis, templated or non-templated enzymatic nucleic acid synthesis, or more specifically, terminal deoxynucleotidyl transferase (TdT) mediated addition of 3′-O-reversibly terminated nucleoside 5′-triphosphates to the 3′-end of 5′-immobilized nucleic acids. During enzymatic nucleic acid synthesis, the following steps are taken on the instrument:
1. Addition solution containing TdT, optionally pyrophosphatase (PPiase), 3′-O-reversibly terminated dNTPs, and required buffer (including salts and necessary reaction components such as metal divalents) is brought to a reaction zone containing an immobilized nucleic acid, where the nucleic acid is immobilized on a surface such as through magnetic beads via a covalent linkage to the 5′ terminus of the nucleic acid. The initial immobilized nucleic acid may be known as an initiator oligonucleotides and comprises N nucleotides, for example 3-100 nucleotides, preferably 10-80 nucleotides, and more preferably 20-65 nucleotides. Initiator oligonucleotides may contain a cleavage site, such as a restriction site or a non-canonical DNA base such as U or 8-oxoG. Addition solution may optionally contain a phosphate sensor, such as E. coli phosphate-binding protein conjugated to MDCC fluorophore, to assess the quality of nucleic acid synthesis as a fluorescent output. dNTPs can be combined in ratios to make DNA libraries, such as NNK syntheses.
2. Wash solution, either in bulk or in discrete droplets, is applied to reaction zones to wash away the addition solution. Wash solution typically has a high solute concentration (>1 M NaCl).
3. Deprotection solution, either in bulk or in discrete droplets, is applied to reaction zones to deprotect the 3′-O-reversible terminator added to the immobilized nucleic acids in the immobilized nucleic acid zone in step I. Deprotection solution typically has a high solute concentration.
4. Wash solution, either in bulk or in discrete droplets, is applied to reaction zones to wash away the deprotection solution.
5. Steps I-IV are repeated until desired sequences are synthesized, for example steps I-IV are repeated 10, 50, 100, 200 or 1000 times.

The present method can be used in the preparation of oligonucleotide sequences, either via synthesis or assembly. The device allows synthesis and movement of defined sequences. Using the present method the initiation sequences can be modified at a specific location above an electrode and the extended oligonucleotides prepared. The initiation sequences at different locations can be exposed to different nucleotides, thereby synthesising different sequences in different regions of the electrokinetic device.

After synthesis of a defined population of different sequences in different regions of the electrokinetic device, the sequences can be further assembled in longer contiguous sequences by joining two or more synthesised strands together.

Described herein is a method for preparing a contiguous oligonucleotide sequence of at least 2n bases in length comprising taking the electrokinetic device as described herein having a plurality of immobilised initiation oligonucleotide sequences, one or more of which contains a cleavage site, using the initiation oligonucleotide sequences to synthesise a plurality of immobilised oligonucleotide sequences of at least n bases in length, using cycles of extension of reversibly blocked nucleotide monomers, selectively cleaving at least two of the immobilised oligonucleotide sequences of least n bases in length into a reaction solution whilst leaving one or more of the immobilised oligonucleotide sequences attached, hybridizing at least two of the cleaved oligonucleotides to each other, to form a splint, and hybridizing one end of the splint to one of the immobilized oligonucleotide sequences and joining at least one of the cleaved oligonucleotides to the immobilised oligonucleotide sequences, thereby preparing a contiguous oligonucleotide sequence of at least 2n bases in length.

The steps of synthesis and assembly may involve high solute concentrations where the ionic strength would degrade the devices without the protecting conformal layer.

The method of moving aqueous droplets may also be used to help facilitate cell-free expression of peptides or proteins. In particular, droplets containing a nucleic acid template and a cell-free system having components for protein expression in an oil-filled environment can be moved using a method of the invention in the described electrokinetic device.

The present invention can be used to automate the movements of droplets in a cartridge. For example, droplets intended for analysis can be moved according to the present invention. The present invention could be incorporated into a cartridge used for local clinician diagnostics. For example it could be used in conjunction with nucleic acid amplification testing (NAAT) to determine nucleic acid targets in, for example, genetic testing for indications such as cancer biomarkers, pathogen testing for example detecting bacteria in a blood sample or virus detection, such as a coronavirus, e.g. SARS-COV-2 for the diagnosis of COVID-19.

The device may be thermocycled to enable nucleic acid amplification, or the device may be held at a desired temperature for isothermal amplification. Having different sequences synthesised in different regions of the device allows multiplex amplification using different primers in different regions of the device.

Furthermore the invention can be used in conjunction with next generation sequencing in which DNA is synthesised by the addition of nucleotides and large numbers of samples are sequenced in parallel. The present invention can be used to accurately locate the individual samples used in next generation sequencing.

The invention can be used to automate library preparation for next generation sequencing. For example the steps of ligation of sequencing adaptors can be carried out on the device.

Amplification of a selective subset of sequences from a sample can then have adaptors attached to enable sequencing of the amplified population.

### Protein Expression Applications

The method of moving aqueous droplets may also be used to help facilitate cell-free expression of peptides or proteins. In particular, droplets containing a nucleic acid template and a cell-free system having components for protein expression in an oil-filled environment can be moved using a method of the invention in the described electrokinetic device.

Disclosed herein is a method for the real-time monitoring of in vitro protein synthesis comprising
1. in vitro transcription and translation of a protein of interest fused to a peptide tag; and
2. monitoring the presence of the peptide tag using a further polypeptide which in the presence of the peptide tag produces a detectable signal.

Disclosed herein is a method for the monitoring of cell free protein synthesis in a droplet on a digital microfluidic device comprising
a. cell free transcription and translation of a protein of interest fused to a peptide tag;

and
b. monitoring the presence of the peptide tag using a further polypeptide which in the presence of the peptide tag produces a detectable signal.

The use of the terms “in-vitro” and “cell free” may be used interchangeably herein.

The detectable signal may be for example fluorescence or luminescence. The detectable signal may also be caused by the binding of a ligand to the complemented oligopeptide, peptide, or polypeptide tag fused to the protein of interest.

The detectable signal may also be caused by the binding of the polypeptide to the protein of interest fused to a His-tag.

Any in vitro transcription and translation may be used, for example extract-based systems derived from rabbit reticulocyte lysate, human lysate, Chinese Hamster Ovary lysate, a wheat germ, HEK293 lysate, E. coli lysate, yeast lysate.

Alternatively the in vitro transcription and translation may be assembled from purified components, for example a system of purified recombinant elements (PURE).

The in vitro transcription and translation may be coupled or uncoupled.

The peptide tag may be one component of a fluorescent protein and the further polypeptide a complementary portion of the fluorescent protein. The fluorescent protein could include sfGFP, GFP, ccGFP, eGFP, deGFP, frGFP, eYFP, eBFP, eCFP, Citrine, Venus, Cerulean, Dronpa, DsRED, mKate, mCherry, mRFP, FAST, SmURFP, miRFP670nano. For example the peptide tag may be GFP₁₁ and the further polypeptide GFP₁₋₁₀. The peptide tag may be one component of sfCherry. The peptide tag may be sfCherry11 and the further polypeptide sfCherry₁₋₁₀. The peptide tag may be CFAST₁₁ or CFAST₁₀ and the further polypeptide NFAST in the presence of a hydroxybenzylidene rhodanine analog.

The peptide tag may also be one component of a protein that forms a detectable substrate, such as a luminescent or colorigenic substrate. The protein could include beta-galactosidase, beta-lactamase, or luciferase.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry11 peptide tags and the synthesis occurs in the presence of multiple sfCherry₁₋₁₀ polypeptides. The protein of interest may be fused to one or more sfCherry11 peptide tags and one or more GFP₁₁ peptide tags and the synthesis occurs in the presence of one or more GFP₁₋₁₀ polypeptides and one or more sfCherry₁₋₁₀ polypeptides.

Any protein of interest may be synthesised. The protein may be an enzyme, for example a terminal deoxynucleotidyl transferase (TdT) enzyme or a truncated version thereof or the homologous amino acid sequence of a terminal deoxynucleotidyl transferase (TdT) enzyme in other species or the homologous amino acid sequence of Polu, Polβ, Polλ, and Polθ of any species or the homologous amino acid sequence of X family polymerases of any species.

Protein expression typically requires an ample supply of oxygen. The most convenient and high yielding way to power CFPS is via oxidative phosphorylation where O₂ serves as the final electron acceptor; however, there are other ways that involve replenishing with energy molecules not involved in oxidative phosphorylation. In a confined microfluidic or digital microfluidic system of droplets, insufficient oxygen is available to enable efficient protein synthesis.

Described herein are improved methods allowing for the cell-free expression of peptides or proteins in a digital microfluidic device. Included is a method for the cell-free expression of peptides or proteins in a microfluidic device wherein the method comprises one or more droplets containing a nucleic acid template (i.e., DNA or RNA) and a cell-free system having components for protein expression in an oil-filled environment, and moving said droplets using electrowetting. The components for the cell-free protein synthesis droplet can be pre-mixed prior to introduction to or mixed on the digital microfluidic device.

The droplet can be repeatedly moved for at least a period of 30 minutes whilst the protein is expressed. The droplet can be repeatedly moved for at least a period of two hours whilst the protein is expressed. The droplet can be repeatedly moved for at least a period of twelve hours whilst the protein is expressed. The act of moving the droplet allows oxygen to be supplied to the droplet and dispersed throughout the droplet. The act of moving improves the level of protein expression over a droplet which remains static.

The droplet can be moved using any means of electrowetting. The droplet can be moved using electrowetting-on-dielectric (EWoD). The electrical signal on the EWoD or optical EWoD device can be delivered through segmented electrodes, active-matrix thin-film transistors, or digital micromirrors.

The filler fluid in the device can be any water immiscible liquid. The filler fluid can be mineral oil, silicone oil such as octamethylcyclotetrasiloxane (CTS), decamethyltetrasiloxane (DMTS) or dodecamethylpentasiloxane (DMPS), an alkyl-based solvent such as decane or dodecane, or a fluorinated oil. The filler fluid can be oxygenated prior to or during the expression process.

A source of supplemental oxygen can be supplied to the droplets. For example droplets or gas bubbles containing gaseous or dissolved oxygen can be merged with the droplets during the protein expression. Additionally, a source of supplemental oxygen can be found by oxygenating the oil that is used as the filler medium. It is well-known in the art that oils such as hexadecane, HFE-7500, and others can be oxygenated to support the oxygen requirements of cell growth, especially E. coli cell growth (RSC Adv., 2017, 7, 40990-40995). Oxygenation can be achieved by aerating the oil with pure oxygen or atmospheric air.

The droplets can be formed before entering the microfluidic device and flowed into the device. Alternatively the droplets can be merged on the device. Included is a method comprising merging a first droplet containing a nucleic acid template such as a plasmid with a second droplet containing a cell-free extract having the components for protein expression to form a combined droplet capable of cell-free protein synthesis.

The droplets can be split on the device either before or after expression. Included herein is a method further comprising splitting the aqueous droplet into multiple droplets. If desired the split droplets can be screened with further additives. Included is a method wherein one or more of the split droplets are merged with additive droplets for screening.

The cell-free expression of peptides or proteins can use a cell lysate having the reagents to enable protein expression. Common components of a cell-free reaction include an energy source, a supply of amino acids, cofactors such as magnesium, and the relevant enzymes. A cell extract is obtained by lysing the cell of interest and removing the cell walls, DNA genome, and other debris by centrifugation. The remains are the cell machinery including ribosomes, aminoacyl-tRNA synthetases, translation initiation and elongation factors, nucleases, etc. Once a suitable nucleic acid template is added, the nucleic acid template can be expressed as a peptide or protein using the cell derived expression machinery.

Any particular nucleic acid template can be expressed using the system described herein. Three types of nucleic acid templates used in CFPS include plasmids, linear expression templates (LETs), and mRNA. Plasmids are circular templates, which can be produced either in cells or synthetically. LETs can be made via PCR. While LETs are easier and faster to make, plasmid yields are usually higher in CFPS. mRNA can be produced through in vitro transcription systems. The methods use a single nucleic acid template per droplet. The methods can use multiple droplets having a different nucleic acid template per droplet.

An energy source is an important part of a cell-free reaction. Usually, a separate mixture containing the needed energy source, along with a supply of amino acids, is added to the extract for the reaction. Common sources are phosphoenolpyruvate, acetyl phosphate, and creatine phosphate. The energy source can be replenished during the expression process by adding further reagents to the droplet during the process.

The cell-free extract having the components for protein expression includes everything required for protein expression apart from the nucleic acid template. Thus the term includes all the relevant ribosomes, enzymes, initiation factors, nucleotide monomers, amino acid monomers, metal ions and energy sources. Once the nucleic acid template is added, protein expression is initiated without further reagents being required.

Thus the cell-lysate can be supplemented with additional reagents prior to the template being added. The cell-free extract having the components for protein expression would typically be produced as a bulk reagent or ‘master mix’ which can be formulated into many identical droplets prior to the distinct template being separately added to separate droplets. Common cell extracts in use today are made from E. coli (ECE), rabbit reticulocytes (RRL), wheat germ (WGE), insect cells (ICE) and Yeast Kluyveromyces (the D2P system). All of these extracts are commercially available.

Rather than originating from a cell extract, the cell-free system can be assembled from the required reagents. Systems based on reconstituted, purified molecular reagents are commercially available, for example the PURE system for protein production, and can be used as supplied. The PURE system is composed of all the enzymes that are involved in transcription and translation, as well as highly purified 70S ribosomes. The protein synthesis reaction of the PURE system lacks proteases and ribonucleases, which are often present as undesired molecules in cell extracts.

Once the CFPS reagents have been enclosed in the droplets, additional reagents can be supplied by merging the original droplet with a second droplet. The second droplet can carry any desired additional reagents, including for example oxygen or ‘power’ sources, or test reagents to which it is desired to expose to the expressed protein.

The droplets can be aqueous droplets. The droplets can contain an oil immiscible organic solvent such as for example DMSO. The droplets can be a mixture of water and solvent, providing the droplets do not dissolve into the bulk filler liquid.

The droplets containing the cell-free extract having the components for protein expression will therefore typically be in the oil filled environment before the nucleic acid templates are added to the droplets. The templates can be added by merging droplets on the microfluidic device. Alternatively, the templates can be added to the droplets outside the device and then flowed into the device for the expression process. For example the expression process can be initiated on the device by increasing the temperature. The expression system typically operates optimally at temperatures above standard room temperatures, for example at or above 29° C.

The expression process typically takes many hours. Thus the process should be left for at least 30 minutes or 1 hour, typically at least 2 hours. Expression can be left for at least 12 hours. During the process of expression the droplets should be moved within the device. The moving improves the process by mixing the reagents and ensuring sufficient oxygen is available within the droplet. The moving can be continuous, or can be repeated with intervening periods of non-movement.

Thus the aqueous droplet can be repeatedly moved for at least a period of 30 minutes or one hour whilst the protein is expressed. The aqueous droplet can be repeatedly moved for at least a period of two hours whilst the protein is expressed. The aqueous droplet can be repeatedly moved for at least a period of twelve hours whilst the protein is expressed. The act of moving the droplet allows mixing within the droplet, and allows oxygen or other reagents to be supplied to the droplet. The act of moving improves the level of protein expression over a droplet which remains static.

The filler fluid in the device can be any water immiscible, non-ionic or hydrophobic liquid. The oil can be mineral oil, silicone oil such as octamethylcyclotetrasiloxane (CTS), decamethyltetrasiloxane (DMTS) or dodecamethylpentasiloxane (DMPS), an alkyl-based solvent such as decane or dodecane, or a fluorinated oil or a mix thereof.

A source of supplemental oxygen can be supplied to the droplets. For example droplets or gas bubbles containing gaseous or dissolved oxygen can be merged with the aqueous droplets during the protein expression. Alternatively the source of oxygen can be a molecular source which releases oxygen. Alternatively the droplets can be moved to an air/liquid boundary to enable increased diffusion of oxygen from a gaseous environment. Alternatively the oil can be oxygenated.

The droplet can be formed before entering the microfluidic device and flowed into the device. Alternatively the droplets can be merged on the device. Included is a method comprising merging a first droplet containing a nucleic acid template such as a plasmid with a second droplet containing a cell-free system having the components for protein expression to form the droplet.

The droplets can be split on the device either before, during or after expression. Included herein is a method further comprising splitting the droplet into multiple droplets. If desired the split droplets can be screened with further additives. Included is a method wherein one of more of the split droplets are merged with additive droplets for screening.

Through an affinity tag, such as a FLAG-tag, HIS-tag, GST-tag, MBP-tag, STREP-tag, or other form of affinity tag, CFPS-expressed proteins can be immobilized to a solid-support affinity resin and fresh batches of CFPS reagent can be delivered over the said resin. Thus, renewed reagents can be used to carry out protein synthesis, closely mimicking industrial methods of continuous flow (CF) and continuous exchange (CE) CFPS. By mimicking CF-and CE-CFPS, users can scale up their CFPS production methods.

Droplets can also contain additives to reduce the effects of biofouling on digital microfluidic surfaces. Specifically, droplets containing CFPS components can also contain additives such as surfactants or detergents to reduce the effects of biofouling on the hydrophobic or superhydrophobic surface of a digital microfluidic device (Langmuir 2011, 27, 13, 8586-8594).

Rather than adding surfactants to the aqueous sample, it is instead possible to add surfactant, such as a sorbitan ester such as Span85 (e.g. Sorbitan trioleate, Sigma Aldrich, SKU 8401240025), to the filler liquid. This has the advantages of enabling CFPS reactions to proceed on-DMF without dilution or adulteration. Additionally, it simplifies the sample preparation procedure for setting up the reactions, increasing the ease of use and the consistency of results. Using 1% w/w Span85 in dodecane allows for dilution-free CFPS reactions on-DMF, as well as dilution-free detection of the expressed non-fluorescent proteins. Other surfactants besides Span85, and oils other than dodecane could be used. A range of concentrations of Span85 could be used. Surfactants could be nonionic, anionic, cationic, amphoteric or a mixture thereof. Oils could be mineral oils or synthetic oils, including silicone oils, petroleum oils, and perfluorinated oils. Surfactants can have a detrimental effect on (1) the CFPS reactions and (2) the efficiency of the detection system (if the detection system involves complementation of a tag and detector). For example, by performing the CFPS reaction on-DMF with oil-surfactant mix, the detection of the expressed protein can also proceed without dilution. It has been shown that surfactants reduce the efficiency of some detection systems, including but not limited to the Split GFP (e.g. GFP₁₁/GFP₁₋₁₀) system, so lowering surfactants from the aqueous reagent mix and instead adding them to the oil can be beneficial.

The peptide tag can be attached to the C or N terminus of the protein. The peptide tag may be one component of a green fluorescent protein (GFP). For example the peptide tag may be GFP₁₁ and the further polypeptide GFP₁₋₁₀. The peptide tag may be one component of sfCherry. The peptide tag may be sfCherry11 and the further polypeptide sfCherry₁₋₁₀.

The protein may be fused to multiple tags. For example the protein may be fused to multiple GFP₁₁ peptide tags and the synthesis occurs in the presence of multiple GFP₁₋₁₀ polypeptides. For example the protein may be fused to multiple sfCherry11 peptide tags and the synthesis occurs in the presence of multiple sfCherry₁₋₁₀ polypeptides. The protein of interest may be fused to one or more sfCherry11 peptide tags and one or more GFP₁₁ peptide tags and the synthesis occurs in the presence of one or more GFP₁₋₁₀ polypeptides and one or more sfCherry₁₋₁₀ polypeptides.

Where used herein “and/or” is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

### Experimental Details

### Adhesion Promotion

Adding 0.5% v/v Silane A-174 to a 1:1 ratio of isopropanol/water and stirring for 30 seconds formed solution 1. Solution 1 was left to stand for at least 2 hours to fully react and was used within 24 hours. Substrates were immersed in the Solution 1 for 30 minutes, while ensuring the flex strips of the TFT arrays were kept dry. Substrates were removed and air dried for 15 minutes and then cleaned in isopropanol for 15-30 seconds with agitation using tweezers. Substrates were dried with an air gun and stored in a Teflon box for Parylene C coating within 30 hours.

### Parylene Coating

Prepared substrates (silanised and non-silanised) were arranged face up on a rotating stage alongside a clean glass slide within the deposition chamber of a thoroughly clean SCS Labcoater 2 and the chamber was sealed. 50 mg of Parylene C dimer was weighed into a disposable aluminium boat and loaded into the sublimation chamber. The system was sealed and pumped down to 50 milliTorr before liquid nitrogen was added to the cold trap. The system continued to evacuate throughout the deposition process. The sublimation chamber was heated to 175° C. and the heater cycled to maintain a target pressure of 0.1 Torr. The sublimation chamber was connected to the deposition chamber by a pyrolysis zone which was heated to 690° C. at a target pressure of 0.5 Torr. The deposition zone remained at ambient temperature, circa 25° C., and around 50 milliTorr. The system was maintained at temperature and pressure for two hours. The system was allowed to return gradually to ambient temperature over 30-40 minutes before the stage and vacuum pump were turned off and the system vented. The samples were removed from the deposition chamber and the coating thickness verified as circa 100 nm by profilometry.

Data shown in Figures shows that in the absence of aqueous surfactant, aqueous droplets are hard to handle, even with surfactant in the oil layer. The absence of or too low a level of aqueous surfactant leads to biofouling and droplets that are difficult to move. Higher levels of surfactant affect the biomolecule interactions needs to applications such as protein expression. The correct balance of the two surfactants is therefore needed in order to achieve protein expression on digital microfluidic devices. Pluronic surfactant F127 gives a good dispense over prolonged time periods. Low levels of pluronic surfactant are compatible with protein expression and detection. Thus Pluronic F127 in the aqueous phase may be used for protein expression applications in droplets on electrowetting devices.

1. A digital microfluidic device comprising a two-dimensional array of planar electrodes wherein the device comprises a population of aqueous droplets containing biomolecules and a first surfactant within a bulk oil phase, wherein the bulk oil phase contains a second surfactant.

2. The device according to claim 1 wherein the aqueous droplets contain a non-ionic surfactant.

3. The device according to claim 1 wherein the aqueous droplets contain a pluronic surfactant.

4. The device according to claim 3 wherein the aqueous droplets contain Pluronic F127.

5. The device according to any one of claims 1 to 3 wherein the oil is mineral oil, silicone oil, an alkyl-based solvent, or a fluorinated oil.

6. The device according to claim 5 wherein the oil is dodecamethylpentasiloxane, decane or dodecane.

7. The device according to any one of claims 1 to 6 wherein the second surfactant is a non-ionic surfactant.

8. The device according to claim 7 wherein the surfactant is a sorbitan ester.

9. The device according to claim 7 wherein the surfactant is Span85.

10. The device according to claim 1 wherein the first surfactant is Pluronic F127 and the second surfactant is Span85.

11. The device according to claim 10 wherein the oil is octamethylcyclotetrasiloxane (CTS), decamethyltetrasiloxane (DMTS) or dodecamethylpentasiloxane.

12. The device according to claim 11 having 0.05% w/w Pluronic F127 in an aqueous buffer in a filler fluid of 0.1% span85 in dodecamethylpentasiloxane (DMPS).

13. The device according to any one of claims 1 to 12 wherein the biomolecules are double stranded nucleic acids or proteins.

14. The device according to any one of claims 1 to 13 wherein the droplets are moved, split or combined using a subset of the electrodes on the device.

15. A method for the cell-free expression of peptides or proteins in a digital microfluidic device according to any one of claims 1 to 14 wherein the method comprises one or more droplets containing a nucleic acid template and a cell-free system having components for protein expression containing a first surfactant in an oil-filled environment, wherein the oil contains a second surfactant, and moving said droplets using electrowetting-on-dielectric (EWoD).

16. The method according to claim 15 wherein the cell-free system is a cell-free extract for protein expression.

17. The method according to claim 15 wherein the cell-free system is prepared from individual reagents.

18. The method according to any one of claims 15 to 17 comprising merging a first droplet containing a nucleic acid plasmid with a second droplet containing a cell-free system having the components for protein expression to form a combined droplet on the microfluidic device.

19. The method according to any one of claims 15 to 18 comprising merging a plurality of first droplets containing a nucleic acid template with a plurality of second droplets containing a cell-free system having the components for protein expression to form multiple combined droplets capable of cell-free protein synthesis.

20. The method according to any one of claims 15 to 19 where the expressed peptides or proteins are detected by optical means.

21. The method for the cell-free expression of peptides or proteins in a digital microfluidic device having an oil-filled environment comprising a second surfactant according to claim 1, the method comprising:
a. taking a plurality of droplets having a different nucleic acid template,
b. taking a plurality of droplets each containing a cell-free system having the components for protein expression,
c. combining the droplets of a. and b. using electrowetting-on-dielectric phenomena to produce a plurality of droplets capable of expressing proteins of different sequence, the droplets containing a first surfactant,
d. mixing the droplets to enable cell-free protein expression, and
e. detecting the expression of proteins within individual droplets.

22. The method according to claims 15 to 21 wherein the first surfactant is Pluronic F127 and the second surfactant is Span85.

23. A kit for preparing a plurality of peptide or proteins comprising
a. a digital microfluidic device;
b. a reagent source to generate a plurality of droplets containing a cell-free system having the components for protein expression and a first surfactant; and
c. an oil, optionally mineral oil, silicone oil, an alkyl-based solvent such as decane or dodecane or a fluorinated oil or a mix thereof, wherein the oil contains a second surfactant.

24. The kit according to claim 23 wherein the cell free system includes ribosomes, enzymes, initiation factors, nucleotide monomers, amino acid monomers, metal ions and energy sources.

25. The kit according to claim 23 or 24, where the first surfactant is Pluronic F127 and the second surfactant is Span85.

26. The kit according to claim 25 having Pluronic F127 in an aqueous buffer and span85 in octamethylcyclotetrasiloxane (CTS), decamethyltetrasiloxane (DMTS) or dodecamethylpentasiloxane (DMPS).
